(19) United States
(12) Patent Application Publication
(10) Pub. No.: US 2011/0177061 A1
(43) Pub. Date: Jul. 21, 2011

(54) METHODS OF TREATING AND PREVENTING NEUROLOGICAL DISORDERS USING DOCOSAHEXAENOIC ACID

(75) Inventors: Paul S. AISEN, Solana Beach, CA (US); Joseph F. Quinn, Portland, OR (US); Karin Yurko-Mauro, Silver Spring, MD (US)

(73) Assignee: MARTEK BIOSCIENCES CORPORATION, Columbia, MD (US)

(21) Appl. No.: 12/833,913

(22) Filed: Jul. 9, 2010

Related U.S. Application Data
(60) Provisional application No. 61/224,836, filed on Jul. 10, 2009.
(60) Provisional application No. 61/359,792, filed on Jun. 29, 2010.

(51) Int. Cl. A61K 31/202; A61K 31/661; A61K 31/232; A61K 31/473; A61K 31/445; A61K 31/27; A61K 31/55; A61K 39/00; A61K 39/395; A61K 31/355; A61P 25/28; A61P 25/00

(52) U.S. Cl. 424/133.1; 514/560; 514/120; 514/547; 514/549; 514/297; 514/319; 514/479; 514/215; 424/184.1; 424/172.1; 424/152.1; 514/458

(57) ABSTRACT

The disclosure relates to methods of treating or preventing neurological disorders using docosahexaenoic acid.

### 1. CROSS REFERENCE TO RELATED APPLICATIONS

This application claims benefit under 35 U.S.C. §119(e) of application Ser. No. 61/224,836, filed Jul. 10, 2009, the contents of which are incorporated herein by reference.

### 2. TECHNICAL FIELD

The disclosure relates to methods of treating and preventing a neurological disorder.

### 3. BACKGROUND

A decline in memory and cognitive function is considered to be a normal consequence of aging in humans. However, as understanding of these physiological and cognitive changes associated with aging are better understood and as the percentage of the population increases in age, more of these changes are considered to be disorders that should be subject to therapeutic intervention. Furthermore, some of these age-related disorders, such as mild cognitive impairment (MCI) and age-associated memory impairment (AAMI) may indicate the early stages of dementia, particularly Alzheimer's disease.

Dementia is characterized by loss of integrated central nervous system functions, resulting in the inability to understand simple concepts or instructions, to store and retrieve information into memory, and in behavioral and personality changes. Commonly used criteria for diagnoses of dementia are provided in the Diagnostic and Statistical Manual for Mental Disorders, American Psychiatric Association, 4th Ed. (DSM-IV). Diagnostic features of dementia according to the DSM-IV include memory impairment and at least one of the following: language impairment (aphasia), lost ability to execute learned motor functions (apraxia), inability to recognize familiar objects (agnosia), or disturbances in executive functioning or decision making.

Dementia of the Alzheimer type (DAT), or simply Alzheimer's Disease (AD) is one of the most prevalent forms of dementia, representing roughly 40 to 60% of diagnosed cases. The disorder typically develops over a period of years, with the affected individual developing cognitive decline over time. People with AD experience memory loss, impairment of decision making and language skills, and develop behavior and personality changes. AD ultimately leads to severe loss of mental capabilities.

Alzheimer's disease can be grouped into early onset and late onset AD. In early onset AD, sometimes referred to as familial AD, the individual develops AD in his/her 30s, 40s, and 50s. This form of AD is uncommon, accounting for about 4-5% of the AD cases. Early onset is associated with certain mutations in several different genes that cause the disease to begin at an earlier age. These include mutations in the genes for presenilin 1, presenilin 2, and amyloid precursor protein (APP).

Late-onset AD, sometimes referred to as sporadic AD, accounts for the majority of AD cases. Its development and pattern of damage in the brain is similar to that of early-onset AD, but late onset AD generally develops in people who are 60 yrs or older. The course of this disease varies from person to person, as does the rate of decline. The causes of late-onset AD are unknown, but they probably include a complex combination of genetic, environmental, and lifestyle factors.

Histologically, AD may be confirmed by physical changes such as the loss of neurons and synapses in the cerebral cortex and certain subcortical regions. This loss results in gross atrophy of the affected regions of the brain. In association with the loss of neurons and synapses, AD is characterized by deposition of abnormal, insoluble extracellular (β-amyloid) and intracellular (tau) proteins.

Current therapies for treating age-related cognitive disorders and dementia, such as AD, include the use of acetylcholinesterase inhibitors and NMDA receptor antagonists. However, these therapies have met with only limited success. Thus, there is a need in the art for new therapeutic approaches for the treatment of age-related cognitive disorders and dementia, such as DAT.

### 4. SUMMARY

In one aspect, the present disclosure provides a method of treating an age-related cognitive disorder, comprising administering to a human subject in need thereof who is identified as being negative for the ApoE4 allele an effective amount of a composition comprising docosahexaenoic acid (DHA) to treat the age-related cognitive disorder.

In some embodiments of treating an age-related disorder, the method may comprise: (a) identifying a human subject negative for the ApoE4 allele, and (b) administering to the human subject in need thereof an effective amount of a composition comprising docosahexaenoic acid (DHA) to treat the age-related cognitive disorder.

Age-related cognitive disorders include mild cognitive impairment (MCI), age-related cognitive decline (ARCD), age-associated memory impairment (AAMI), and age-associated cognitive impairment (AACI).

In another aspect, the present disclosure provides a method of treating dementia, comprising: administering to a human subject in need thereof who is identified as being negative for the ApoE4 allele an effective amount of a composition comprising docosahexaenoic acid (DHA) to treat dementia.

In some embodiments of treating dementia, the method may comprise: (a) identifying a human subject negative for the ApoE4 allele, and (b) administering to the human subject in need thereof an effective amount of a composition comprising docosahexaenoic acid (DHA) to treat dementia.

In another aspect, the present disclosure provides a method of treating Alzheimer's disease, comprising: administering to a human subject in need thereof who is identified as being negative for the ApoE4 allele an effective amount of a composition comprising docosahexaenoic acid (DHA) to treat Alzheimer's disease.

In some embodiments of treating Alzheimer's disease, the method may comprise: (a) identifying a human subject negative for the ApoE4 allele; and (b) administering to the human subject in need thereof an effective amount of a composition comprising docosahexaenoic acid (DHA) to treat Alzheimer's disease.

In some embodiments, the human subject suffers from mild to moderate Alzheimer's disease. In some embodiments, the human subject suffers from mild Alzheimer's disease. In some embodiments, the human subject has a mini-mental state examination (MMSE) score of <26. In some embodiments, the human subject has a MMSE score from 10 to 26, more particularly from 14 to 26. In some embodiments, the subjects have an MMSE score in the range of 20 to 26, more particularly from 21 to 26.

Generally, in the methods described herein, the composition has a docosahexaenoic acid (DHA) to eicosapentaenoic acid (EPA) ratio of higher than 4:1 wt/wt. In some embodiments of the method, the DHA to EPA ratio is at least 5:1 wt/wt, at least 10:1 wt/wt, at least 20:1 wt/wt, at least 50:1 wt/wt, or at least 100:1 wt/wt. In some embodiments, the DHA to EPA ratio is about 16:1 wt/wt. In some embodiments, the composition of DHA is substantially free of EPA. In some embodiments, the composition of DHA has no EPA. The DHA may be in the form of a phospholipid, triglyceride, free fatty acid, or in the form of an alkyl ester, such as methyl, ethyl, propyl or butyl ester.

The DHA may be obtained or derived from any source, such as fish oil, plant oil, nut oil, or oil from an organism genetically modified to synthesize DHA. In some embodiments, the DHA is a microbial oil or is derived from microbial oil. Microbial oil includes those derived from oleaginous microorganisms, such as microorganisms of the genus Crypthecodinium, Schizochytrium, or Thraustochytrium.

The DHA may be in any form including: a highly purified algal oil comprising the DHA, a plant oil comprising DHA, triglyceride oil comprising the DHA, phospholipids comprising the DHA, a combination of protein and phospholipids comprising the DHA, dried marine microalgae comprising the DHA, sphingolipids comprising the DHA, esters of the DHA, free fatty acid, a conjugate of the DHA with another bioactive molecule, and combinations thereof. Long chain fatty acids can be provided in amounts and/or ratios that are different from the amounts or ratios that occur in the natural source of the fatty acids, such as by blending, purification, enrichment and genetic engineering of the source. Bioactive molecules can include any suitable molecule, including, but not limited to, a protein, an amino acid (e.g., naturally occurring amino acids such as DHA-glycine, DHA-lysine, or amino acid analogs), a drug, and a carbohydrate. The forms outlined herein allow flexibility in the formulation of foods with high sensory quality, dietary supplements, medical foods, and pharmaceutical agents.

In some embodiments, the DHA may be administered adjunctively with another anti-Alzheimer's therapy, i.e., the composition of DHA and the anti-Alzheimer's therapy may be administered sequentially or simultaneously. The anti-Alzheimer's therapy may be administered before or after administration of DHA.

Any Alzheimer's therapy known or to be developed, including any anti-Alzheimer's drug, may be used in the methods of the invention. In some embodiments, the anti-Alzheimer's therapy is a drug selected from an acetylcholinesterase inhibitor, an NMDA receptor antagonist, a vaccine (e.g., amyloid vaccine), an antibody against the β-amyloid protein (e.g., a human or humanized monoclonal antibody), a β or γ secretase inhibitor or a tau inhibitor

In some embodiments, the composition of DHA may be administered adjunctively with other non-DHA therapies, including compounds or compositions, that have a therapeutic benefit for treating an age-related cognitive disorder, dementia, or AD. In some embodiments, the composition of DHA may be administered to a subject adjunctively with an anti-inflammatory agent, including nonsteroidal anti-inflammatory drugs (NSAIDs), steroidal anti-inflammatory drugs, or cholesterol lowering agents.

In the methods described herein, the human subject negative for the ApoE4 allele may carry the ApoE2 or ApoE3 allele. In some embodiments, the subject to be treated is homozygous for the ApoE2 or ApoE3 allele.

In some embodiments, the DHA is administered in a therapeutically effective amount to a human subject to treat an age-related cognitive disorder, dementia, or Alzheimer's disease. In some embodiments, the DHA may be administered in an amount of from about 1.5 mg per kg body weight per day to about 125 mg per kg body weight per day. In some embodiments, the DHA is administered in an amount of from about 150 mg to about 10 g per day, from about 0.5 g per day to about 5 g per day; or from about 1 g per day to about 5 g per day. In some embodiments, the DHA is administered in an amount of about 1 g per day.

The DHA may be administered in varying treatment regimens, including administration at least once per day, at least twice per day, or at least two times weekly. The treatment may last for periods of at least 6 months, at least 1 yr, at least 1.5 yrs, at least 2 yrs, at least 5 yrs, or until time in which a therapeutic benefit is achieved.

The DHA composition may be administered in the form of a capsule, gel, or tablet, particularly through oral administration.

### 5. BRIEF DESCRIPTION OF THE FIGURES

FIG. 1 provides the co-primary outcome using the Alzheimer's Disease Assessment Score (“ADAS”)-cog test for subjects treated with DHA and placebo, where the plot shows a modest difference in 12 month ADAS scores (Wilcoxan rank p=0.027), but no significant difference between DHA and placebo groups using the linear-mixed-effect (“LME”) model, and no difference in sensitivity (Generalized Estimating Equations “GEE,” Analysis of Covariance “ANCOVA”) and per protocol analyses.

FIG. 2 provides co-primary outcome using the Clinical Dementia Rating Sum of Boxes (“CDR-SOB”) score for assessing AD, where the results show no significant difference between DHA and placebo groups using the LME model, GEE, or ANCOVA on Intention to Treat (ITT) or per protocol populations, with adjustment for Mini-Mental State Examination (MMSE) and gender.

FIG. 3 provides the secondary outcome using Alzheimer's Disease Cooperative Study-Activities of Daily Living (ADCS-ADL) for assessing AD, showing that there was no significant difference between DHA and placebo groups using LME model, GEE, or ANCOVA on ITT or per protocol populations, with adjustment for MMSE and gender.

FIG. 4 provides the secondary outcome using Neuropsychiatric Inventory (NPI) for assessing AD, showing that there was no significant difference between DHA and placebo groups using LME model, GEE, or ANCOVA on ITT or per protocol populations, with adjustment for MMSE and gender.

FIG. 5 provides the secondary outcome using MMSE for assessing AD, showing that there was no significant difference between DHA and placebo groups after 18 months of treatment; and

FIGS. 6A and 6B provide the results of pre-specified sub-group analysis of clinical data using ADAS-cog for assessing AD, showing a statistically significant difference in the effect of DHA administration between subjects positive for the ApoE4 allele (FIG. 6A) and those who were negative for the ApoE4 allele (FIG. 6B).

FIGS. 7A and 7B provide the results of analysis of ADAS-cog scores in ApoE negative subjects having MMSE scores of ≦21 (FIG. 7A) and ApoE4 negative subjects having an MMSE score of >21 showing that the mildly impaired group (MMSE>21) having a significantly less decline when compared to the subjects with an MMSE score of ≦21.

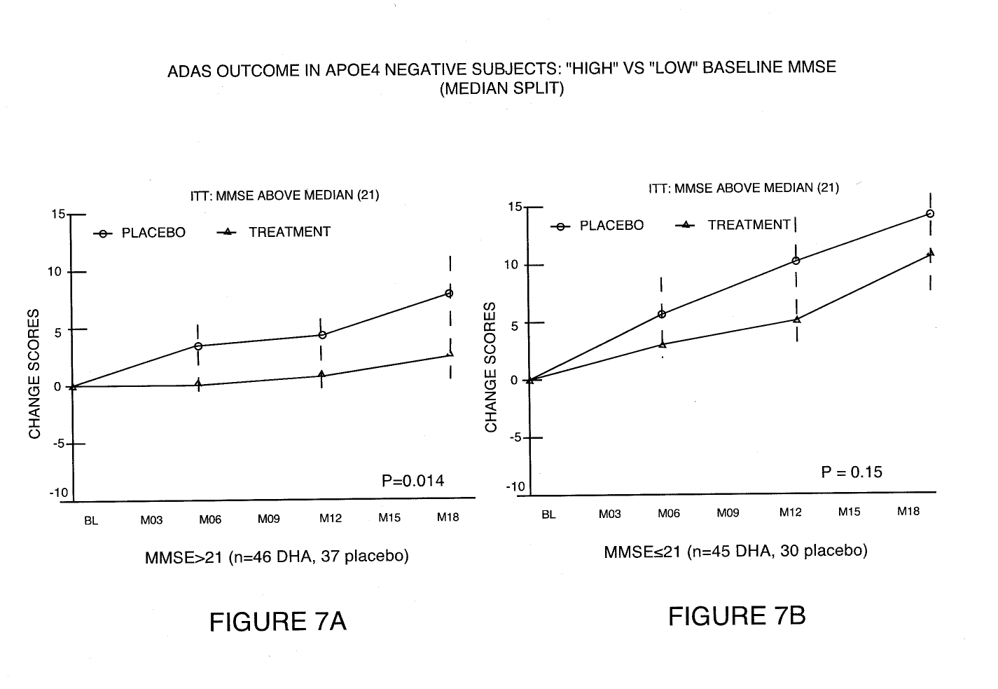

### 6. DETAILED DESCRIPTION

For the descriptions herein and the appended claims, the singular forms “a”, “an” and “the” include plural referents unless the context clearly indicates otherwise. Thus, for example, reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

In reference to the present disclosure, the technical and scientific terms used in the descriptions herein will have the meanings commonly understood by one of ordinary skill in the art, unless specifically defined otherwise.

The present disclosure shows that human subjects who are afflicted with AD and are negative for the ApoE4 allele derive significant benefit from administration of DHA in reducing the characteristic decline in cognitive ability associated with AD. A post hoc analysis indicates that the benefit is more pronounced in the ApoE population having and MMSE score of 21 to 26.

Human Apolipoprotein E or ApoE is a 299 amino acid polypeptide that acts as a ligand for low-density lipoprotein receptors, mediating the transport of cholesterol and other lipoproteins throughout the body. Human ApoE protein is encoded by a gene located on chromosome 19. ApoE is primarily expressed in hepatic parenchymal cells in the liver. The second largest area of ApoE expression is in the brain, with astrocytes being the major site of production. In the brain, ApoE protein is associated with enhancing proteolytic break-down of β-amyloid peptide, both within and between cells.

The apoE gene exists in three commonly occurring alleles, denoted e2 (apoE2), e3 (apoE3), and e4 (apoE4). The e2 allele encodes for the protein isoform ApoE2 in which the amino acid at position 112 is cysteine and the amino acid at position 158 is cysteine. Thus, as used herein “ApoE2 allele” refers to the gene encoding the polypeptide isoform of ApoE in which the amino acid at position 112 is cysteine and the amino acid at position 158 is cysteine. The e3 allele is the most common allele, encoding for the protein isoform ApoE3 in which the amino acid at position 112 is cysteine and the amino acid at position 158 is arginine. Thus, as used herein, the term “ApoE3 allele” refers to the gene encoding a polypeptide isoform of ApoE in which the amino acid at position 112 is cysteine and the amino acid at position 158 is arginine. The e4 allele encodes a polypeptide isoform of ApoE in which the amino acid at position 112 is arginine and the amino acid at position 158 is arginine. Thus, as used herein, the “ApoE4 allele” refers to the gene encoding a polypeptide isoform of ApoE in which the amino acid at position 112 is arginine and the amino acid at position 158 is arginine. The nucleotide and amino acid sequences of ApoE, ApoE2, ApoE3 and ApoE4 are well-known in the art. For example, the human apoE4 gene has the Genbank accession number of M10065.

Accordingly, in one aspect, the present disclosure provides a method of treating an age-related cognitive disorder, the method comprising administering to a human subject in need thereof who is identified as being negative for the ApoE4 allele an effective amount of a composition of docosahexaenoic acid (DHA) to treat the age-related cognitive disorder. In some embodiments, the method of treating an age-related cognitive disorder, comprises: (a) identifying a human subject negative for the ApoE4 allele, and (b) administering to the human subject in need thereof an effective amount of a composition comprising docosahexaenoic acid (DHA) to treat the age-related cognitive disorder.

As used herein, “age-related cognitive disorder” encompasses a constellation of disorders that includes mild cognitive impairment (MCI), age-related cognitive decline (ARCD), and age-associated memory impairment (AAMI), sometimes referred to as age-associated cognitive impairment (AACI).

“Mild Cognitive Impairment” or MCI″ refers to a cognitive disorder which is diagnosed where there is evidence of memory impairment beyond that expected for a subject's age and education, but where general cognitive and function abilities are maintained and there is an absence of diagnosed dementia (see Winblad et al., 2004, “Mild cognitive impairment—beyond controversies, towards a consensus,” J Intern Med. 256:240-246; Petersen et al., 2004, “Mild cognitive impairment as a diagnostic entity,” J Intern Med. 256:183-194). A diagnostic algorithm for diagnosing MCI can be found in Peterson and Negash, 2008, CNS Spectr. 13(1):45-53) as well as in Diagnostic and Statistical Manual of Mental Disorders, 4th Ed. (DSM-IV), incorporated herein by reference. There is evidence suggesting that while amnestic MCI patients may not meet neuropathologic criteria for Alzheimer's disease, patients may be in a transitional stage of evolving Dementia, such as Alzheimer's disease, such that MCI, when memory loss is the predominant symptom (amnestic MCI), is a risk factor for Alzheimer's disease. When individuals have impairments in domains other than memory, it is classified as non-amnestic single- or multiple-domain MCI and these individuals are believed to be more likely to convert to other dementias (i.e., dementia with Lewy bodies).

“Age-related cognitive decline” or “ARCD” refers to an age-related cognitive disorder in which the subject experiences deterioration in memory and learning, attention and concentration, thinking, use of language, and other mental functions but is otherwise healthy (see, e.g., Levy, 1994, “Aging-associated cognitive decline,” Int Psychogeriatr 1994; 6:63-8). Diagnostic criteria of ARCD can be found in DSM-IV. ARCD usually occurs gradually and can be characterized physiologically by decrease in brain mass with age and decrease in synaptic density. However, symptoms are not sufficiently severe to be diagnosed as dementia or Alzheimer's disease.

“Age-associated memory impairment” or “AAMI” or “age-associated cognitive impairment” or AACI” refers to an age-related cognitive disorder in which the subject is impaired in tests assessing memory, but also in tests of executive functions associated with frontal lobe function. Diagnostic criteria for AAMI and AACI includes the presence of self-reported memory decline, evidence of memory loss as determined by performance on a standardized memory test, adequate intellectual functioning and absence of dementia or other memory affecting diseases, such as stroke; and age of 50 yrs or older. Diagnostic criteria of AAMI can be found in DSM-IV and Barker et al., 1995, Br J Psychiatry. 167(5):642-8. This evaluation is generally complemented by an individual's symptoms; the rate of symptom onset; presentation of symptoms; and progression of symptoms over time. The population of subjects diagnosed with AAMI includes those with very early dementia (e.g., pre-dementia).

In the methods herein, a human subject diagnosed with any one of the age-related cognitive disorders can be treated by administering a composition of DHA, as further described in detail below.

In another aspect, the methods herein relate to use of DHA to treat dementia based on a subject's ApoE allelic status. Accordingly, in some embodiments, the method for treating dementia can comprise administering to a human subject in need thereof who is identified as being negative for the ApoE4 allele an effective amount of a composition of docosahexaenoic acid (DHA) to treat dementia. In some embodiments, the method for treating dementia, comprises: (a) identifying a human subject negative for the ApoE4 allele; and (b) administering to the human subject in need thereof an effective amount of a composition comprising docosahexaenoic acid (DHA) to treat dementia.

As used herein, “dementia” refers to a group of disorders characterized by a global deterioration of intellectual functioning in clear consciousness, and is characterized by one or more symptoms of disorientation, impaired memory, impaired judgment, and/or impaired intellect. DSM-IV defines “dementia” as characterized by multiple cognitive deficits that include impairments in memory and lists various dementias according to presumed etiology. The DSM-IV sets forth a generally accepted standard for such diagnosing, categorizing and treating of dementia and associated psychiatric disorders, including vascular dementia and multi-infarct dementia.

“Vascular disease” or “Dementia associated with or caused by vascular diseases,” generally refers to cerebrovascular diseases (e.g., infarctions of the cerebral hemispheres), which generally have a fluctuating course with periods of improvement and stepwise deterioration. “Vascular dementia” can include one or more symptoms of disorientation, impaired memory and/or impaired judgment. Vascular dementia can be caused by discrete multiple infarctions, or other vascular causes including, for example, autoimmune vasculitis, such as that found in systemic lupus erythematosus; infectious vasculitis, such as Lyme's disease; recurrent intracerebral hemorrhages; and stroke. Human subjects diagnosed with these subgroups of dementia may also be treated in accordance with the methods of the invention by administration of DHA.

In another aspect, DHA can be administered to treat Alzheimer's disease based on a subject's ApoE allelic status. Accordingly, in some embodiments, the method of treating Alzheimer's disease can comprise administering to a human subject in need thereof who is identified as being negative for the ApoE4 allele an effective amount of a composition of docosahexaenoic acid (DHA) to treat Alzheimer's disease. In some embodiments, the method of treating or preventing Alzheimer's disease, comprises: (a) identifying a human subject negative for the ApoE4 allele; and (b) administering to the human subject in need thereof an effective amount of a composition comprising docosahexaenoic acid (DHA) to treat Alzheimer's disease.

“Alzheimer's disease”, “AD”, “Dementia of Alzheimer's Type” or “DAT” refers to a progressive neurologic disease of the brain that leads to the irreversible loss of neurons and dementia. The clinical hallmarks of Alzheimer's disease are progressive impairment in memory, judgment, decision making, orientation to physical surroundings, and language. A working diagnosis of Alzheimer disease is usually made on the basis of the neurologic examination, such as that provided in DSM-IV. These neurological assessments can be supplemented by other diagnostic procedures, such as medical imaging techniques and the detection of tau protein and/or β-amyloid protein, as further described below.

In some embodiments, the human subject to be treated is diagnosed as having from mild to moderate Alzheimer's disease. In some embodiments, the human subject to be treated is diagnosed as having mild Alzheimer's disease. As further discussed below, the mini-mental state examination (MMSE) can be used to assess the severity of cognitive impairment in Alzheimer's disease. In some embodiments, the human subject to be treated has a MMSE score of ≦26. In some embodiments, the human subject to be treated has a MMSE score from about 10 to 26, more particularly from about 14 to 26. In some embodiments, the subject to be treated has a MMSE score in the range of about 20 to 26, more particularly from about 21 to 26.

In the methods herein, a human subject suffering from the above disorders can be treated by administering a composition comprising DHA. As used herein, “DHA” refers to docosahexaenoic acid, also known by its chemical name (all-Z)-4,7,10,13,16,19-docosahexaenoic acid, as well as any salts or derivatives thereof. Thus, the term “DHA” encompasses the free acid DHA as well as DHA alkyl esters and triglycerides containing DHA. DHA is an ω-3 polyunsaturated fatty acid. Hence, in various embodiments, the DHA used in the method may be in the form of a phospholipid, a triglyceride, free fatty acid, or an alkyl ester. In some embodiments, the alkyl ester may comprise DHA methyl ester, ethyl ester, or propyl ester, as further described below.

Any source of DHA can be used in the compositions and methods described herein, including, for example, animal, plant and microbial sources. In some embodiments, a source of oils containing DHA suitable for the compositions and methods described herein is an animal source. Examples of animal sources include aquatic animals (e.g., fish, marine mammals; crustaceans such as krill and other euphausids; rotifers, etc.) and lipids extracted from animal tissues (e.g., brain, liver, eyes, etc.) and animal products such as eggs or milk. Examples of plant sources include macroalgae, flaxseeds, rapeseeds, corn, evening primrose, soy and borage. Examples of microorganisms include microalgae, protists, bacteria and fungi (including yeast). For example, the DHA may be purified from fish oil, plant oil, seed oil, or other naturally occurring oils such that the DHA to EPA ratio are within the scope described herein.

In some embodiments, the composition of DHA is a microbial oil or is derived from microbial oil. Exemplary microbes from which microbial oil may be obtained, include, among others, the microbial groups Stramenopiles, Thraustochytrids, and Labrinthulids. Stramenopiles includes microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), and Chlamydomyxa* (species include labyrinthuloides, montana) (*=there is no current general consensus on the exact taxonomic placement of these genera).

In some embodiments, the microbial oil source is oleaginous microorganisms, such as certain marine algae. As used herein, “oleaginous microorganisms” are defined as microorganisms capable of accumulating greater than 20% of the dry weight of their cells in the form of lipids. In some embodiments, the DHA is derived from a phototrophic or heterotrophic single cell organism or multicellular organism, e.g., an algae. For example, the DHA may be derived from a diatom, e.g., a marine dinoflagellates (algae), such as Crypthecodinium sp., Thraustochytrium sp., Schizochytrium sp., or combinations thereof. Exemplary samples of C. cohnii, have been deposited with the American Type Culture Collection at Rockville, Md., and assigned the accession numbers 40750, 30021, 30334-30348, 3054130543, 30555-30557, 30571, 30572, 30772-30775, 30812, 40750, 50050-50060, and 50297-50300.

As used herein, the term microorganism, or any specific type of organism, includes wild strains, mutants or recombinant types. Organisms which can produce an enhanced level of oil containing DHA are considered to be within the scope of this invention. For example, cultivation of dinoflagellates such as C. cohnii has been described previously. See, e.g., U.S. Pat. No. 5,492,938 and Henderson et al., Phytochemistry 27:1679-1683 (1988). Also included are microorganisms designed to efficiently use more cost-effective substrates while producing the same amount of DHA as the comparable wild-type strains.

Organisms useful in the production of DHA can also include any manner of transgenic or other genetically modified organisms, such as a genetically modified plant or a genetically modified microorganism manipulated to produce DHA. e.g., plants, grown either in culture fermentation or in crop plants, e.g., cereals such as maize, barley, wheat, rice, sorghum, pearl millet, corn, rye and oats; or beans, soybeans, peppers, lettuce, peas, Brassica species (e.g., cabbage, broccoli, cauliflower, brussel sprouts, rapeseed, and radish), carrot, beets, eggplant, spinach, cucumber, squash, melons, cantaloupe, sunflowers, safflower, canola, flax, peanut, mustard, rapeseed, chickpea, lentil, white clover, olive, palm, borage, evening primrose, linseed, and tobacco. In some embodiments, the DHA is derived from a soybean source, including wild type and genetically modified soybean sources.

In some embodiments, the DHA may be purified in the form of free fatty acids, fatty acid esters, phospholipids, triglycerides, diglycerides, monoglycerides or combinations thereof by any means known to those of skill in the art. In some embodiments, the DHA comprises an ester. The term “ester” refers to the replacement of the hydrogen in the carboxylic acid group of the DHA molecule with another substituent. Typical esters are known to those in the art, a discussion of which is provided by Higuchi, T. and V. Stella in “Pro-drugs as Novel Delivery Systems,” Vol. 14, A.C.S. Symposium Series, Bioreversible Carriers in Drug Design, Ed. Edward B. Roche, American Pharmaceutical Association, Pergamon Press, 1987, and Protective Groups in Organic Chemistry, McOmie ed., Plenum Press, New York, 1973. In some embodiments, the ester is an alkyl ester. Examples of more common esters include C₁-C₆ esters, e.g., methyl, ethyl, propyl, butyl, pentyl, hexyl, or branched variations thereof, e.g., isopropyl, isobutyl, isopentyl, or t-butyl. In some embodiments, the ester is a carboxylic acid protective ester group, esters with aralkyl (e.g., benzyl, phenethyl), esters with lower alkenyl (e.g., allyl, 2-butenyl), esters with lower-alkoxy-lower-alkyl (e.g., methoxymethyl, 2-methoxyethyl, 2-ethoxyethyl), esters with lower-alkanoyloxy-lower-alkyl (e.g., acetoxymethyl, pivaloyloxymethyl, 1-pivaloyloxyethyl), esters with lower-alkoxycarbonyl-lower-alkyl (e.g., methoxycarbonylmethyl, isopropoxycarbonylmethyl), esters with carboxy-lower alkyl (e.g., carboxymethyl), esters with lower-alkoxycarbonyloxy-lower-alkyl (e.g., 1-(ethoxycarbonyloxy)ethyl, 1-(cyclohexyloxycarbonyloxy)ethyl), esters with carbamoyloxy-lower alkyl (e.g., carbamoyloxymethyl), and the like. In some embodiments, the added substituent is a cyclic hydrocarbon group, e.g., C₁-C₆ cycloalkyl, or C₁-C₆ aryl ester. Other esters include nitrobenzyl, methoxybenzyl, benzhydryl, and trichloroethyl. In some embodiments, the ester substituent is added to a DHA free acid molecule when the DHA is in a purified or semi-purified state. Alternatively, the DHA ester is formed upon conversion of a triglyceride to a ester. One of skill in the art can appreciate that some non-esterified DHA molecules can be present in the DHA compositions, e.g., DHA molecules that have not been esterified, or DHA triglyceride ester linkages that have been cleaved, e.g., hydrolyzed. In some embodiments, the non-esterified DHA molecules or the DHA triglyceride molecules constitute less than 3% (mol/mol), about 0.01% to about 2% (mol/mol), about 0.05% to about 1% (mol/mol), or about 0.01% to about 0.5% (mol/mol) of the total DHA molecules. In some embodiments, the amount of ethyl ester of DHA in the compositions may be at least about 91, 92, 93, 94, 95, 96, 97, 98, or 99 wt. %.

In some embodiments, the DHA of the present invention is a triglyceride, diglyceride or monoglyceride. A “triglyceride” is a glyceride in which the glycerol is esterified with three fatty acid groups. Typical triglycerides are known to those in the art. In some embodiments, the DHA is in the form of a triglyceride or a diglyceride, wherein one or more fatty acid groups other than DHA are present in the triglyceride or diglyceride. In some embodiments, DHA is the only fatty acid group on a triglyceride or diglyceride molecule. In some embodiments, one or more fatty acid groups of a triglyceride have been hydrolyzed, or cleaved.

In some embodiments, the DHA of the present invention is in the form of free fatty acid. “Free fatty acid” refers to fatty acid compounds in their acidic state, and salt derivatives thereof.

In the embodiments described herein, the composition of DHA for use in the methods may be obtained by standard techniques known in the art. In some embodiments, EPA may be removed during the purification of DHA, or alternatively, the DHA may be from an organism that produces DHA with the levels of EPA described herein, for example a production organism is selected that produces DHA with an insubstantial amount of EPA. DHA can be purified to various levels. DHA purification can be achieved by any means known to those of skill in the art, and can include the extraction of total oil from an organism which produces DHA. In some embodiments, EPA, ARA, and/or DPAn6 are then removed from the total oil, for example, via chromatographic methods. Alternatively, DHA purification can be achieved by extraction of total oil from an organism which produces DHA, but produces little, if any, amount of EPA, ARA, DPAn6, and/or flavonoids. In some embodiments, the oil can be diluted with other oils, such as sunflower oil to achieve the desired concentration of fatty acids.

Microbial oils useful in the present invention can be recovered from microbial sources by any suitable means known to those in the art. For example, the oils can be recovered by extraction with solvents such as chloroform, hexane, methylene chloride, methanol and the like, or by supercritical fluid extraction. Alternatively, the oils can be extracted using extraction techniques, such as are described in U.S. Pat. No. 6,750,048 and International Pub. No. WO 2001/053512, both filed Jan. 19, 2001, and entitled “Solventless extraction process,” both of which are incorporated herein by reference in their entirety. Processes for the preparation of various forms of DHA are also described in, among others, US Patent Publication No. 2009/0023808 “Production and Purification of Esters of Polyunsaturated Fatty Acids” by Raman et al., and US Patent Publication No. 2007/0032548 “Polyunsaturated fatty acids for treatment of dementia and pre-dementia-related conditions” by Ellis, incorporated herein by reference.

Additional extraction and/or purification techniques are taught in International Pub. No. WO 2001/076715; International Pub. No. WO 2001/076385; U.S. Pub. No. 2007/0004678; U.S. Pub. No. 2005/0129739; U.S. Pat. No. 6,399,803; and International Pub. No. WO 2001/051598; all of which are incorporated herein by reference in their entirety. The extracted oils can be evaporated under reduced pressure to produce a sample of concentrated oil material. Processes for the enzyme treatment of biomass for the recovery of lipids are disclosed in International Pub. No. WO 2003/092628; U.S. Pub. No. 2005/0170479; EP Pat. Pub. 0776356 and U.S. Pat. No. 5,928,696, the last two entitled “Process for extracting native products which are not water-soluble from native substance mixtures by centrifugal force,” all of which are incorporated herein by reference in their entirety.

In some embodiments, the DHA can be prepared as esters using a method comprising: a) reacting a composition comprising polyunsaturated fatty acids in the presence of an alcohol and a base to produce an ester of a polyunsaturated fatty acid from the triglycerides; and b) distilling the composition to recover a fraction comprising the ester of the polyunsaturated fatty acid, optionally wherein the method further comprises: c) combining the fraction comprising the ester of the polyunsaturated fatty acid with urea in a medium; d) cooling or concentrating the medium to form a urea-containing precipitate and a liquid fraction; and e) separating the precipitate from the liquid fraction. See, e.g., U.S. patent publication no. US2009/0023808, incorporated by reference herein in its entirety. In some embodiments, the purification process includes starting with refined, bleached, and deodorized oil (RBD oil), then performing low temperature fractionation using acetone to provide a concentrate. The concentrate can be obtained by base-catalyzed transesterification, distillation, and silica refining to produce the final DHA product.

Methods of determining purity levels of fatty acids are known in the art, and may include, e.g., chromatographic methods such as, e.g., HPLC silver ion chromatographic columns. Alternatively, purity levels may be determined by gas chromatography, with or without converting DHA to the corresponding alkyl ester. The percentage of fatty acids may also be determined using Fatty Acid Methyl Ester (FAME) analysis.

In some embodiments, the DHA esters can be derived from undiluted oil from a single cell microorganism, and in some embodiments, from undiluted DHASCO-T® (Martek Biosciences Corporation, Columbia, Md.). In some embodiments, the oil from which DHA compositions can be derived includes single cell microorganism oils that are manufactured by a controlled fermentation process followed by oil extraction and purification using methods common to the vegetable oil industry. In certain embodiments, the oil extraction and purification steps can include refining, bleaching, and deodorizing. In some embodiments, the undiluted DHA oil comprises about 40% to about 50% DHA by weight (about 400-500 mg DHA/g oil). In certain embodiments, the undiluted DHA oil can be enriched by cold fractionation (resulting in oil containing about 60% wt/wt of DHA triglyceride), which DHA fraction optionally can be transesterified, and subjected to further downstream processing to produce the active DHA of the invention. In some embodiments of the invention, downstream processing of the oil comprises distillation and/or silica refinement.

Thus, to produce oil from which DHA can be derived, in certain aspects, the following steps can be used: fermentation of a DHA producing microorganism; harvesting the biomass; spray drying the biomass; extracting oil from the biomass; refining the oil; bleaching the oil; chill filtering the oil; deodorizing the oil; and adding an antioxidant to the oil. In some embodiments, the microorganism culture can be progressively transferred from smaller scale fermenters to a production size fermenter. In some embodiments, following a controlled growth over a pre-established period, the culture can be harvested by centrifugation then pasteurized and spray dried. In certain embodiments, the dried biomass can be flushed with nitrogen and packaged before being stored frozen at −20° C. In certain embodiments, the DHA oil can be extracted from the dried biomass by mixing the biomass with n-hexane or isohexane in a batch process which disrupts the cells and allows the oil and cellular debris to be separated. In certain embodiments, the solvent can then be removed.

In some embodiments, the crude DHA oil can then undergo a refining process to remove free fatty acids and phospholipids. The refined DHA oil can be transferred to a vacuum bleaching vessel to assist in removing any remaining polar compounds and pro-oxidant metals, and to break down lipid oxidation products. The refined and bleached DHA oil can undergo a final clarification step by chilling and filtering the oil to facilitate the removal of any remaining insoluble fats, waxes, and solids.

Optionally, the DHA can be deodorized under vacuum in a packed column, counter current steam stripping deodorizer. Antioxidants such as ascorbyl palmitate, alpha-tocopherol, and tocotrienols can optionally be added to the deodorized oil to help stabilize the oil. In some embodiments, the final, undiluted DHA oil is maintained frozen at −20° C. until further processing.

In some embodiments, the DHA oil can be converted to DHA ester by methods known in the art. In some embodiments, DHA esters of the invention can be produced from DHA oil by the following steps: cold fractionation and filtration of the DHA oil (to yield for example about 60% triglyceride oil); direct transesterification (to yield about 60% DHA ethyl ester); molecular distillation (to yield about 88% DHA ethyl ester); silica refinement (to yield about 90% DHA ethyl ester); and addition of an antioxidant.

In some embodiments, the cold fractionation step can be carried out as follows: undiluted DHA oil (triglyceride) at about 500 mg/g DHA is mixed with acetone and cooled at a controlled rate in a tank with −80° C. chilling capabilities. Saturated triglycerides crystallize out of solution, while polyunsaturated triglycerides at about 600 mg/g DHA remain in the liquid state. The solids containing about 300 mg/g can be filtered out with a 20 micron stainless steel screen from the liquid stream containing about 600 mg/g DHA. The solids stream can then be heated (melted) and collected. The 600 mg/g DHA liquid stream can be desolventized with heat and vacuum and then transferred to the transesterification reactor.

In some embodiments, the transesterification step is carried out on the 600 mg/g DHA oil, wherein the transesterification is done via direct transesterification using ethanol and sodium ethoxide. The transesterified material (DHA-ethyl ester) can then be subject to molecular distillation and thus, further distilled (3 passes, heavies, lights, heavies) to remove most of the other saturated fatty acids and some sterols and non-saponifiable material. The DHA-ethyl ester (DHA-EE) can be further refined by passing it through a silica column.

DHA free fatty acids can be made using, for example, the DHA containing oils described above. In some embodiments, the DHA-FFA can be obtained from DHA esters. DHA triglycerides, for example, can be saponified followed by a urea adduction step to make free fatty acids.

In some embodiments of the method, the DHA composition used has a level of DHA that is at least 40 wt % of total wt of fatty acid content. In some embodiments, the weight % of the DHA in the composition of DHA is at least 50 wt % of total wt of fatty acid content, at least 60 wt % of total wt of fatty acid content; at least 70 wt % of total wt of fatty acid content; at least 80 wt % of total wt of fatty acid content; at least 85 wt % of total wt of fatty acid content; at least 90 wt % of total wt of fatty acid content; at least 95 wt % of total wt of fatty acid content; at least 96 wt % of total wt of fatty acid content; at least 97 wt % of total wt of fatty acid content; at least 98 wt % of total wt of fatty acid content; or at least 99 wt % of total wt of fatty acid content.

In some embodiments, DHA is present in an amount of about 35% to about 99.9% (wt/wt) of the total fatty acid content of the dosage form or unit dose, about 40% to about 99% (wt/wt) of the total fatty acid content of the dosage form or unit dose, about 45% to about 98% (wt/wt) of the total fatty acid content of the dosage form or unit dose, about 65% to about 99.9% (wt/wt) of the total fatty acid content of the dosage form or unit dose, or about 85% to about 95% (wt/wt) of the total fatty acid content of the dosage form or unit dose. In some embodiments, the DHA is present in an amount greater than about 65% (wt/wt) of the total fatty acid content of the dosage form or unit dose, greater than about 85% (wt/wt) of the total fatty acid content of the dosage form or unit dose, greater than about 90% (wt/wt) of the total fatty acid content of the dosage form or unit dose, or greater than about 95% (wt/wt) of the total fatty acid content of the dosage form or unit dose. In some embodiments, the oil can be diluted with other oils, such as sunflower oil, to achieve the desired concentration of fatty acids.

In some embodiments, the DHA is about 30% (wt/wt) or more of the total fatty acid content of the dosage form or unit dose, about 30% to about 99.9% (wt/wt) of the total fatty acid content of the dosage form or unit dose, about 35% to about 99.9% (wt/wt) of the total fatty acid content of the dosage form or unit dose, about 35% to about 60% (wt/wt) of the total fatty acid content of the dosage form or unit dose, about 35% to about 50% (wt/wt) of the total fatty acid content of the dosage form or unit dose, about 37% to about 45% (wt/wt) of the total fatty acid content of the dosage form or unit dose, or about 38% to about 43% (wt/wt) of the total fatty acid content of the dosage form or unit dose. In some embodiments, the DHA is greater than about 35%, about 37%, about 38%, about 39% or about 40% (wt/wt) of the total fatty acid content of the dosage form or unit dose. In some embodiments, the DHA is about 30% to about 99.5% (wt/wt) of the total fatty acid content of the dosage form or unit dose, or about 40% to about 65% (wt/wt) of the total fatty acid content of the dosage form or unit dose.

In some of these embodiments, the DHA comprises about 40% to about 45% (wt/wt) of the total fatty acid content of the dosage form or unit dose. In some of these embodiments, the DHA comprises about 35% to about 45% (wt/wt) of the total fatty acid content of the dosage form or unit dose. In some of embodiments, the DHA comprises about 55% to about 67% (wt/wt) of the total fatty acid content of the dosage form or unit dose. In some embodiments, the DHA comprises greater than about 70% (wt/wt) of the total fatty acid content of the dosage form or unit dose. In some embodiments, the DHA comprises about 85% to about 99.5% (wt/wt) of the total fatty acid content of the dosage form or unit dose.

In some embodiments, the DHA is greater than about 80% (wt/wt) of the total fatty acid content of the dosage form or unit dose, about 80% to 99.9% (wt/wt) of the total fatty acid content of the dosage form or unit dose, about 85% to about 99% (wt/wt) of the total fatty acid content of the dosage form or unit dose, about 87% to about 98% (wt/wt) of the total fatty acid content of the dosage form or unit dose, or about 90% to about 97% (wt/wt) of the total fatty acid content of the dosage form or unit dose. In some embodiments, the DHA is great than about 95%, about 97%, about 98%, about 99% or about 99.5% (wt/wt) of the total fatty acid content of the dosage form or unit dose.

In some embodiments, the DHA comprises about 35% to about 96% of the weight of the dosage form or unit dose. In some embodiments, the DHA comprises about 38% to about 42% of the weight of the dosage form or unit dose. In some embodiments, the DHA in the dosage form or unit dose comprises about 35% to about 45% of the total weight of the dosage form or unit dose. In some embodiments, the DHA in the dosage form or unit dose comprises about 55% of the total weight of the dosage form or unit dose. In some embodiments, the DHA in the dosage form or unit dose comprises about 85% to about 96% of the total weight of the dosage form or unit dose.

In some embodiments, the DHA is about 30% (wt/wt) or more of the total oil content of the dosage form or unit dose, about 30% to about 99.9% (wt/wt) of the total oil content of the dosage form or unit dose, about 35% to about 99.9% (wt/wt) of the total oil content of the dosage form or unit dose, about 35% to about 60% (wt/wt) of the total oil content of the dosage form or unit dose, about 35% to about 50% (wt/wt) of the total oil content of the dosage form or unit dose, about 37% to about 45% (wt/wt) of the total oil content of the dosage form or unit dose, or about 38% to about 43% (wt/wt) of the total oil content of the dosage form or unit dose. In some embodiments, the DHA is greater than about 35%, about 37%, about 38%, about 39% or about 40% (wt/wt) of the total oil content of the dosage form or unit dose. In some embodiments, the DHA is about 30% to about 99.5% (wt/wt) of the total oil content of the dosage form or unit dose, or about 40% to about 65% (wt/wt) of the total oil content of the dosage form or unit dose.

In some of these embodiments, the DHA comprises about 40% to about 45% (wt/wt) of the total oil content of the dosage form or unit dose. In some of these embodiments, the DHA comprises about 35% to about 45% (wt/wt) of the total oil content of the dosage form or unit dose. In some of embodiments, the DHA comprises about 55% to about 67% (wt/wt) of the total oil content of the dosage form or unit dose. In some embodiments, the DHA comprises greater than about 70% (wt/wt) of the total oil content of the dosage form or unit dose. In some embodiments, the DHA comprises about 85% to about 99.5% (wt/wt) of the total oil content of the dosage form or unit dose.

In some embodiments, the DHA is greater than about 80% (wt/wt) of the total oil content of the dosage form or unit dose, about 80% to 99.9% (wt/wt) of the total oil content of the dosage form or unit dose, about 85% to about 99% (wt/wt) of the total oil content of the dosage form or unit dose, about 87% to about 98% (wt/wt) of the total oil content of the dosage form or unit dose, or about 90% to about 97% (wt/wt) of the total oil content of the dosage form or unit dose. In some embodiments, the DHA is greater than about 95%, about 97%, about 98%, about 99% or about 99.5% (wt/wt) of the total oil content of the dosage form or unit dose. With respect to comparison of DHA to total fatty acid content or total oil content, weight % can be determined by calculating the area under the curve (AUC) using standard means, e.g., dividing the DHA AUC by the total fatty acid AUC.

As used herein, “or less” or “less than about” refers to percentages that include 0%, or amounts not detectable by current means. As used herein, “max” refers to percentages that include 0%, or amounts not detectable by current means.

In some embodiments, the composition of DHA used in the methods has a DHA to eicosapentaenoic acid (EPA) ratio that is higher than 4:1 wt/wt. The term “EPA” refers to eicosapentaenoic acid, known by its chemical name (all Z) 5,8,11,14,17-eicosapentaenoic acid, as well as any salts or derivatives thereof. Thus, the term “EPA” encompasses the free acid EPA as well as EPA alkyl esters and triglycerides containing EPA. EPA is an ω-3 polyunsaturated fatty acid. Typical content of omega-3 fatty acids found in fatty fish have a ratio of DHA to EPA ratio of 4:1 or less, wt/wt.

In some embodiments of the method, the composition of DHA has a DHA to EPA ratio which is at least 5:1 wt/wt, at least 6:1 wt/wt, 7:1 wt/wt, at least 8:1 wt/wt, at least 9:1 wt/wt, at least 10:1 wt/wt, at least 12:1 wt/wt, at least 14:1 wt/wt, at least 16:1 wt/wt, at least 18:1 wt/wt, at least 20:1 wt/wt, at least 40:1 wt/wt, at least 60:1 wt/wt, at least 80:1 wt/wt, at least 100:1 wt/wt, or higher. In some embodiments of the method, the composition of DHA has a DHA to EPA ratio of about 10:1 wt/wt, 12:1 wt/wt, 14:1 wt/wt, 16:1 wt/wt, 18:1 wt/wt, 20:1 wt/wt, 40:1 wt/wt, 60:1 wt/wt, 80:1 wt/wt, or 100:1 wt/wt.

In some embodiments, the composition of DHA is substantially free of EPA. As used herein, a composition of DHA that is “substantially free of EPA” refers to a preparation of DHA in which EPA is less than 3% of the total fatty acid content of the composition, less than 2% of the total fatty acid content of the composition, less than 1% of the total fatty acid content of the composition, less than 0.5% of the total fatty acid content of the composition, less than 0.2% of the total fatty acid content of the composition, or less than 0.01% of the total fatty acid content of the composition. In some embodiments, the EPA is not detectable in the composition using techniques known in the art. In some embodiments, the DHA composition has no EPA.

DHA can also be administered substantially free of arachidonic acid (ARA). ARA refers to the compound (all-Z)-5,8,11,14-eicosatetraenoic acid (also referred to as (5Z,8Z,11Z,14Z)-icosa-5,8,11,14-tetraenoic acid), as well as any salts or derivatives thereof. Thus, the term “ARA” encompasses the free acid ARA as well as ARA alkyl esters and triglycerides containing ARA. ARA is an ω-6 polyunsaturated fatty acid. DHA is “substantially free of ARA” when ARA is less than about 3% (wt/wt) of the total fatty acid content of the dosage form. In some embodiments, ARA comprises less than about 2% (wt/wt) of the total fatty acid content of the dosage form, less than 1% (wt/wt) of the total fatty acid content of the dosage form, less than 0.5% (wt/wt) of the total fatty acid content of the dosage form, less than 0.2% (wt/wt) of the total fatty acid content of the dosage form, or less than 0.01% (wt/wt) of the total fatty acid content of the dosage form. In some embodiments, the dosage form has no detectable amount of ARA.

DHA can also be administered substantially free of docosapentaenoic acid 22:5 n-6 (DPAn6). The term “DPAn6” refers to docosapentaenoic acid, omega 6, known by its chemical name (all-Z)-4,7,10,13,16-docosapentaenoic acid, as well as any salts or esters thereof. The term “DPAn6” encompasses the free acid DPAn6 as well as DPAn6 alkyl esters and triglycerides containing DPAn6. DPAn6 is an ω-6 polyunsaturated fatty acid. DHA is “substantially free of DPAn6” when DPAn6 is less than about 3% (wt/wt) of the total fatty acid content of the dosage form. In some embodiments, DPAn6 comprises less than about 2% (wt/wt) of the total fatty acid content of the dosage form, less than 1% (wt/wt) of the total fatty acid content of the dosage form, less than 0.5% (wt/wt) of the total fatty acid content of the dosage form, less than 0.2% (wt/wt) of the total fatty acid content of the dosage form, or less than 0.01% (wt/wt) of the total fatty acid content of the dosage form. In some embodiments, the dosage form has no detectable amount of DPAn6.

In some embodiments, the dosage form of the present invention does not contain a measurable amount of docosapentaenoic acid 22:5n-3 (DPAn3); docosapentaenoic acid 22:5n-6 (DPAn6); and/or 4,7,10,13,16,19,22,25 octacosaoctaenoic acid (C28:8).

In some embodiments, the DHA is administered in the substantial absence of therapeutic levels of uridine and its pharmaceutically acceptable salts (e.g., uridine monophosphate). In some embodiments, the DHA is administered with less than 100 mg, more particularly less than 10 mg, more particularly less than 5 mg and more particularly less that 1 mg of uridine and its pharmaceutically acceptable salts. In some embodiments, the DHA is administered with no detectable amount of uridine.

In some embodiments, the DHA is administered in the substantial absence of therapeutic levels of choline. In some embodiments, the DHA is administered with less than 100 mg, more particularly less than 10 mg, more particularly less than 5 mg and more particularly less that 1 mg of choline. In some embodiments, the DHA is administered with no detectable amount of choline.

In some embodiments, the composition of DHA may include an additional lipid. As used herein, the term “lipid” includes phospholipids (PL); free fatty acids; esters of fatty acids; triacylglycerols (TAG); diacylglycerides; monoacylglycerides; phosphatides; waxes (esters of alcohols and fatty acids); sterols and sterol esters; carotenoids; xanthophylls (e.g., oxycarotenoids); hydrocarbons; and other lipids known to one of ordinary skill in the art. The lipid can be chosen to have minimal adverse health effects or minimally affect the effectiveness of DHA when administered in combination with DHA.

In some embodiments, the composition of DHA may include an additional unsaturated lipid. In some embodiments, the unsaturated lipid is a polyunsaturated lipid, such as an omega-3 fatty acid or omega-6 fatty acid. An exemplary omega-6 fatty acid that may be used in the composition is docosapentaenoic acid (DPA), including DPA (n-6) or DPA (n-3).

In the methods and compositions herein, additional fatty acids can be present in the dosage form or unit dose or composition. These fatty acids can include fatty acids that were not removed during the purification process, i.e., fatty acids that were co-isolated with DHA from an organism. In some embodiments, one or more non-DHA fatty acids can be added to the dosage form or unit dose to achieve a desired concentration of specific non-DHA fatty acids. Any of these fatty acids can be present in various concentrations. For example, in some embodiments, the dosage form or unit dose comprises 0.01% to about 4% (wt/wt) of oleic acid. In some embodiments, the dosage form or unit dose comprises 0.01% to 0.5% (wt/wt) of one or more of the following fatty acids: (a) capric acid; (b) lauric acid; (c) myristic acid; (d) palmitic acid; (e) palmitoleic acid; (f) heptadecanoic acid; (g) stearic acid; (h) oleic acid; (i) linoleic acid; (j) α-linolenic acid; (k) arachidic acid; (l) eicosenoic acid; (m) arachidonic acid; (n) erucic acid; (o) docosapentaenoic acid 22:5n-3 (DPAn3); and (p) nervonic acid. In some embodiments, a dosage form or unit dose comprises 0.01% to 0.1% (wt/wt) of one or more of the following fatty acids: (a) lauric acid; (b) heptadecanoic acid; (c) stearic acid; (d) arachidic acid; (e) eicosenoic acid; and (f) arachidonic acid. In some embodiments, a dosage form or unit dose comprises less than 0.5% (wt/wt) each of the following fatty acids: (a) capric acid; (b) lauric acid; (c) myristic acid; (d) palmitic acid; (e) palmitoleic acid; (f) heptadecanoic acid; (g) stearic acid; (h) linoleic acid; (i) α-linolenic acid; (j) arachidic acid; (k) eicosenoic acid; (l) arachidonic acid; (m) erucic acid; (n) docosapentaenoic acid 22:5n-3 (DPAn3); and (o) nervonic acid. In some embodiments, the dosage form or unit doses of the present invention do not contain a measurable amount of one or more of the following fatty acids: (a) capric acid; (b) linoleic acid; (c) α-linolenic acid; and (d) docosapentaenoic acid 22:5n-3 (DPAn3).

In some embodiments, the dosage form or unit dose comprises 0.1% to 60% (wt/wt) of one or more of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3 (DPAn3); (k) docosapentaenoic acid 22:5n-6 (DPAn6); and (k) 4,7,10,13,16,19,22,25 octacosaoctaenoic acid (C28:8). In some embodiments, the dosage form or unit dose comprises 20% to 40% (wt/wt) of one or more of the following fatty acids, or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; j) docosapentaenoic acid 22:5n-3 (DPAn3); (k) docosapentaenoic acid 22:5n-6 (DPAn6); and (l) 4,7,10,13,16,19,22,25 octacosaoctaenoic acid (C28:8). In some embodiments, the dosage form or unit dose comprises less than 1% (wt/wt) each of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3 (DPAn3); (k) docosapentaenoic acid 22:5n-6 (DPAn6); and (l) 4,7,10,13,16,19,22,25 octacosaoctaenoic acid (C28:8).

In some embodiments the dosage form comprises 0.1% to 20% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; G) DPA n-3 (22:5, n-3); (k) DPA n-6 (22:5, n-6); and (l) 4,7,10,13,16,19,22,25 octacosaoctaenoic acid (C28:8). In some embodiments, the dosage form comprises 1% to 5% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) DPA n-3 (22:5, n-3); (k) DPA n-6 (22:5, n-6); and (l) 4,7,10,13,16,19,22,25 octacosaoctaenoic acid (C28:8). In some embodiments, the dosage form comprises less than 1% each of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8).

In some of embodiments of DHA dosage form described herein, the dosage form is characterized by one or more the following fatty acids (or esters thereof). The embodiments provided herein may further comprise about 2% or less (wt/wt) of capric acid (C10:0). The embodiments herein may further comprise about 6% or less (wt/wt) of lauric acid (C12:0). The embodiments herein may further comprise about 20% or less (wt/wt), or about 5% to about 20% (wt/wt) of myristic acid (C14:0). The embodiments herein may further comprise about 20% (wt/wt) or less, or about 5% to about 20% (wt/wt) of palmitic acid (C16:0). The embodiments herein may further comprise about 3% (wt/wt) or less of palmitoleic acid (C16:1n-7). The embodiments herein may further comprise about 2% (wt/wt) or less of stearic acid (C18:0). The embodiments herein may further comprise about 40% (wt/wt) or less, or about 10% to about 40% (wt/wt) of oleic acid (C18:1n-9). The embodiments herein may further comprise about 5% (wt/wt) or less of linoleic acid (C18:2). The embodiments herein may further comprise about 2% (wt/wt) or less of nervonic acid (C24:1). The embodiments herein may further comprise about 3% (wt/wt) or less of other fatty acids or esters thereof. The DHA dosage form with the preceding characteristics may comprise DHASCO®, an oil derived from Crypthecodinium cohnii containing docosahexaenoic acid (DHA).

An exemplary DHA (triglyceride) containing oil derived from Crypthecodinium cohnii is characterized by the specified amount of components listed in Table 1, where “Max” refers to the amount of the component that can be present up to the specified amount.

TABLE 1

| | CONCENTRATION (WT/WT) |
|---|---|
| FATTY ACIDS | |
| 10:0 | MAX 2% |
| 12:0 | MAX 6% |
| 14:0 | 5%-20% |
| 16:0 | 5%-20% |
| 16:1 | MAX 3% |
| 18:0 | MAX 2% |
| 18:1 | 10%-40% |
| 18:2 | MAX 5% |
| 22:6 DHA | 40% TO 45% |
| 24:1 | MAX 2% |
| OTHERS | MAX 3% |
| ELEMENTAL COMPOSITION | |
| ARSENIC | MAX 0.5 PPM |
| COPPER | MAX 0.1 PPM |
| IRON | MAX 0.5 PPM |
| LEAD | MAX 0.2 PPM |
| MERCURY | MAX 0.04 PPM |
| PHOSPHOROUS | MAX 10 PPM |
| CHEMICAL CHARACTERISTICS | |
| PEROXIDE VALUE | MAX 5 MEQ/KG |
| FREE FATTY ACID | MAX 0.4% |
| UNSAPONIFIABLE MATTER | MAX 3.5% |

An exemplary undiluted DHA (triglyceride) containing oil derived from Crypthecodinium cohnii is characterized by amount of DHA described herein, and one or more, or all of the features listed below in Table 2, where “Max” refers to the amount of the component that can be present up to the specified amount.

TABLE 2
Characteristics of Undiluted DHA Oil

| TEST | SPECIFICATION |
|---|---|
| DHA CONTENT MG/DHA/G OIL | MIN 480 MG/G |
| FREE FATTY ACID | MAX. 0.4% |
| PEROXIDE VALUE (PV) | MAX. 5 MEQ/KG |
| ANISIDINE VALUE (AV) | MAX 20 |
| MOISTURE AND VOLATILES (M & V) | MAX. 0.02% |
| UNSAPONIFIABLE MATTER | MAX. 3.5% |
| INSOLUBLE IMPURITIES | MAX. 0.1% |
| TRANS FATTY ACID | MAX. 1% |
| ARSENIC | MAX. 0.5 PPM |
| CADMIUM | MAX. 0.2 PPM |
| CHROMIUM | MAX. 0.2 PPM |
| COPPER | MAX. 0.1 PPM |
| IRON | MAX. 0.5 PPM |
| LEAD | MAX. 0.2 PPM |
| MANGANESE | MAX. 0.04 PPM |
| MERCURY | MAX. 0.04 PPM |
| MOLYBDENUM | MAX. 0.2 PPM |
| NICKEL | MAX. 0.2 PPM |
| PHOSPHORUS | MAX. 10 PPM |
| SILICON | MAX. 500 PPM |
| SULFUR | MAX. 100 PPM |
| 18:1 N-9 OLEIC ACID | MAX. 10% |
| 20:5 N-3 EPA | MAX. 0.1% |
| UNKNOWN FATTY ACIDS | MAX. 3.0% |

In some embodiments, an oil is characterized by one or more the following fatty acids (or esters thereof), expressed as wt % of the total fatty acid content. The embodiments provided herein may further comprise about 2% or less (w/w) of capric acid (C10:0). The embodiments provided herein may further comprise about 6% or less (w/w) of lauric acid (C12:0). The embodiments provided herein may further comprise about 20% or less, or about 10 to about 20% (w/w) of myristic acid (C14:0). The embodiments provided herein may further comprise about 15% or less, or about 5 to about 15% (w/w) of palmitic acid (C16:0). The embodiments provided herein may further comprise about 5% or less (w/w) of palmitoleic acid (C16:1n-7). The embodiments provided herein may further comprise about 2% or less (w/w) of stearic acid (C18:0). The embodiments provided herein may further comprise about 20% or less, or about 5% to about 20% (w/w) of oleic acid (C18:1n-9). The embodiments provided herein may further comprise about 2% or less (w/w) of linoleic acid (C18:2). The embodiments provided herein may further comprise about 2% or less (w/w) of nervonic acid (C24:1). The embodiments provided herein may further comprise about 3% or less (w/w) of other fatty acids. An oil with the preceding characteristics may be an oil derived from Crypthecodinium cohnii containing docosahexaenoic acid (DHA).

In some embodiments, the dosage form comprises, measured in percentage of free fatty acid, about 35-65%, 40-55%, 35-57%, or 57-65% DHA (22:6 n-3); about 0-2% capric acid (10:0); about 0-6% lauric acid (12:0); about 10-20% myristic acid (14:0); about 5-15% palmitic acid (16:0); about 0-5% palmitoleic acid (16:1); about 0-2% stearic acid (18:0); about 5-20% or 5-25% oleic acid (18:1); about 0-2% linoleic acid (18:2); and about 0-2% nervonic acid (24:1, n-9). In one embodiment, such an oil is from a microorganism of the genus Thraustochytrium. In another embodiment, the free fatty acid content is less than 0.4%.

The present invention also provides compositions comprising at least about 40 wt. % DHA and at least about 0.1 wt % of DPA (n-3). In some embodiments, the compositions comprise at least about 55, 56, 57, 58, 59, 60, 61, 62, 63, 64, 65 wt. % DHA, optionally in triglyceride form, as a percentage of total fatty acids.

An exemplary DHA containing oil derived from Crypthecodinium cohnii is characterized by the specified amount of components listed in Table 3, where “Max” refers to the amount of the component that can be present up to the specified amount.

TABLE 3

| | CONCENTRATION (WT/WT) |
|---|---|
| FATTY ACIDS | |
| 10:0 | 0-2% |
| 12:0 | 0-6% |
| 14:0 | 10%-20% |
| 16:0 | 5%-15% |
| 16:1 | 0-5% |
| 18:0 | 0-2% |
| 18:1 | 5%-20% |
| 18:2 | 0-2%% |
| 22:6 (N-3) DHA | 57%-65% |
| 24:1 | 0-2% |
| OTHERS | 0-3% |
| ELEMENTAL COMPOSITION | |
| ARSENIC | MAX 0.5 PPM |
| COPPER | MAX 0.1 PPM |
| IRON | MAX 0.5 PPM |
| LEAD | MAX 0.2 PPM |
| MERCURY | MAX 0.2 PPM |
| PHOSPHOROUS | MAX 10 PPM |
| CHEMICAL CHARACTERISTICS | |
| PEROXIDE VALUE | MAX 5 MEQ/KG |
| FREE FATTY ACID | MAX 0.4% |
| UNSAPONIFIABLE MATTER | MAX 3.5% |
| TRANS FATTY ACIDS | <3.5% |
| MOISTURE AND VOLATILES | <0.1% |
| INSOLUBLE IMPURITIES | <0.1% |

In some embodiments, an oil is characterized by one or more the following fatty acids (or esters thereof), expressed as wt % of the total fatty acid content. The embodiments provided herein may further comprise about 0.1% or less (w/w) of myristic acid (C14:0) or is not detectable. The embodiments provided herein may further comprise about 0.5% or less (w/w) of palmitic acid (C16:0). The embodiments provided herein may further comprise about 0.5% or less (w/w) of palmitoleic acid (C16:1n-7). The embodiments provided herein may further comprise about 0.5% or less (w/w) of stearic acid (C18:0), or is not detectable. The embodiments provided herein may further comprise about 4% or less (w/w) of oleic acid (C18:1n-9). The embodiments provided herein may further comprise less than 0.1% (w/w) of linoleic acid (C18:2) or is not detectable. The embodiments provided herein may further comprise less than 0.1% (w/w) of eicosapentaenoic acid (C20:5) or is not detectable. The embodiments provided herein may further comprise about 2% or less (w/w) of decosapentaenoic acid (22:5n-3). The embodiments provided herein may further comprise about 1% or less (w/w) of octacosaoctaenoic acid (28:8 n-3). The embodiments provided herein may further comprise about 0.5% or less (w/w) of tetracosaenoic acid (24:1n9). The embodiments provided herein may further comprise about 1% or less (w/w) of other fatty acids. The DHA in oil with the preceding characteristics may be in the form of a DHA ester, preferably an alkyl ester, such as a methyl ester, ethyl ester, propyl ester, or combinations thereof, prepared from an algal oil prepared from the Crypthecodinium, cohnii sp.

In some embodiments, the DHA composition may comprise DHASCO®. DHASCO® is an oil derived from Crypthecodinium cohnii containing high amounts of docosahexaenoic acid (DHA), and more specifically contains the following approximate exemplary amounts of these fatty acids, as a percentage of the total fatty acids: myristic acid (14:0) 10-20%; palmitic acid (16:0) 10-20%; palmitoleic acid (16:1) 0-2%; stearic acid (18:0) 0-2%; oleic acid (18:1) 10-30%; linoleic acid (18:2) 0-5%; arachidic acid (20:0) 0-1%; behenic acid (22:0) 0-1%; docosapentaenoic acid (22:5) 0-1%; docosahexanoic acid (22:6) (DHA) 40-45%; nervonic acid (24:1) 0-2%; and others 0-3%.

The present invention also provides compositions comprising at least about 40 wt. % DHA and at least about 0.1 wt. % of 4,7,10,13,16,19,22,25 octacosaoctaenoic acid (C28:8). In some embodiments, the compositions comprise at least about 55, 56, 57, 58, 59, 60, 61, 62, 63, 64, 65 wt. % DHA, optionally in triglyceride form, as a percentage of total fatty acids. In other embodiments, the compositions comprise at least about 90, 91, 92, 93, 94, 95, 96, 97, 98, or 99 wt. % of DHA, optionally in ethyl ester form, as a percentage of total fatty acids. In certain embodiments, the amount of C28:8 in the compositions may be at least about 0.2, 0.3, 0.4, 0.5, 0.6, 0.7, 0.8, 0.9, 1.0, 1.1, 1.2, 1.3, 1.4 or 1.5 wt. %. The C28:8 may be present in any form, including triglyceride or ester form. For example, the C28:8 may be present in ethyl ester form.

In other embodiments, the compositions comprise at least about 90, 91, 92, 93, 94, 95, 96, 97, 98, or 99 wt. % of DHA, optionally in ethyl ester form, as a percentage of total fatty acids. In certain embodiments, the amount of DPA (n-3) in the compositions may be at least about 0.1, 0.2, 0.3, 0.4, 0.5, 0.6, 0.7, 0.8, 0.9, or 1.0 wt. % of DPA (n-3). The DPA (n-3) may be present in triglyceride or ester form. For example, the DPA (n-3) may be present in ethyl ester form. In certain embodiments, the compositions comprise all three of the DHA, C28:8 and DPA (n-3) in the concentration ranges specified above.

In further embodiments, the compositions may comprise less than about 1.0, 0.9, 0.8. 0.7, 0.6, 0.5, 0.4, 0.3, 0.2, or 0.1 wt. % EPA in addition to the DHA and C28:8. In one embodiment, the compositions may comprise less than about 0.25 wt. % EPA. The EPA may be present in any form, including triglyceride or ester form. In some embodiments, the compositions may comprise 0 wt. % EPA.

The present invention also provides compositions comprising at least about 90 wt. % of DHA and at least one additional fatty acid or a derivative thereof. In some embodiments, the amount of DHA in the compositions may be at least about 91, 92, 93, 94, 95, 96, 97, 98, or 99 wt. %. In certain embodiments, the additional fatty acid may have a boiling point of about 150-170° C. at a pressure of 0.8 mm Hg.

An exemplary DHA-containing oil derived from the algal oil of Crypthecodinium Cohnii, wherein the DHA comprises an ethyl ester, can be characterized by the specified amount of components listed in Table 4, where “Max” refers to the amount of the component that can be present up to the specified amount.

TABLE 4

| | |
|---|---|
| DHA CONTENT (MG/G) | 855-945 |
| FATTY ACID CONTENT: % OF TOTAL EE | |
| EICOSAPENTAENOIC ACID (20:5ω3) | ND |
| MYRISTIC ACID (14:0) | 0.1% |
| PALMITIC ACID (16:0) | 0.5% |
| PALMITOLEIC ACID (16:1ω7) | 0.4% |
| STEARIC ACID (18:0) | ND |
| OLEIC ACID (18:1ω9) | 4% |
| LINOLEIC ACID (18:2ω6) | ND |
| DOCOSAPENTAENOIC ACID (22:5ω3) | 1.3% |
| OCTACOSAOCTAENOIC ACID (28:8ω3) | 0.9% |
| TETRACOSAENOIC ACID (24:1ω9) | 0.3% |
| OTHERS | 1.1% |
| ELEMENTAL COMPOSITION | |
| ARSENIC | MAX 0.5 PPM |
| COPPER | MAX 0.1 PPM |
| IRON | MAX 0.5 PPM |
| LEAD | MAX 0.2 PPM |
| MERCURY | MAX 0.04 PPM |
| CHEMICAL CHARACTERISTICS | |
| PEROXIDE VALUE | MAX 10.0 MEQ/KG |

ND = NOT DETECTABLE

In some embodiments, an oil is characterized by one or more the following fatty acids (or esters thereof), expressed as wt % of the total fatty acid content. The embodiments provided herein may further comprise about 12% or less, or about 6% to about 12% (w/w) of myristic acid (C14:0). The embodiments provided herein may further comprise about 28% or less, or about 18 to about 28% (w/w) of palmitic acid (C16:0). The embodiments provided herein may further comprise about 2% or less (w/w) of stearic acid (C18:0). The embodiments provided herein may further comprise about 8% or less of (w/w) oleic acid (C18:1n-9). The embodiments provided herein may further comprise about 2% or less (w/w) of linoleic acid (C18:2). The embodiments provided herein may further comprise about 2% or less (w/w) of arachidonic acid (C20:4). The embodiments provided herein may further comprise about 3% or less (w/w) of eicosapentaenoic acid (C20:5). The embodiments provided herein may further comprise about 18% or less, or about 12% to about 18% (w/w) of decosapentaenoic acid (22:5n-6). The embodiments provided herein may further comprise about 10% or less (w/w) of other fatty acids. In some of these embodiments, the ratio of wt % of DHA to wt % of DPAn6 is about 2.5 to about 2.7. An oil with the preceding characteristics may comprise Life's DHA™ (also formerly referenced as DHA™-S and DHASCO®-S), Martek Biosciences, Columbia, Md.), an oil derived from the Thraustochytrid, Schizochytrium sp., that contains a high amount of DHA and also contains docosapentaenoic acid (n-6) (DPAn-6).

In some embodiments, more specifically, DHA™-S contains the following approximate exemplary amounts of these fatty acids, as a percentage of total fatty acids: myristic acid (14:0) 8.71%; palmitic acid (16:0) 22.15%; stearic acid (18:0) 0.66%; linoleic acid (18:2) 0.46%; arachidonic acid (20:4) 0.52%; eicosapentenoic acid (20:5, n-3) 1.36%; docosapentaenoic acid (22:5, n-6) (DPAn-6) 16.28%; docosahexaenoic acid (DHA) (22:6, n-3) 41.14%; and others 8%.

In some embodiments, the dosage form comprises, measured in percentage of free fatty acid, about 35-45% DHA (22:6 n-3); about 0-2% lauric acid (12:0); about 5-10% myristic acid (14:0); about 5-20% palmitic acid (16:0); about 0-5% palmitoleic acid (16:1); about 0-5% stearic acid (18:0); about 0-5% vaccenic acid or oleic acid (18:1n-7 and n-9, respectively); about 0-2% linoleic acid (18:2, n-6); about 0-5% stearidonic acid (18:4 n-3); about 0-10% 20:4 n-3, n-5, or n-6; about 0-2% adrenic acid 22:4 n-6; about 0-5% DPA n-3 (22:5); about 10-25% DPA n-6 (22:5); and 0-2% 24:0. In one embodiment, such an oil is from a microorganism of the genus Schizochytrium.

An exemplary DHA (triglyceride) containing oil derived from Schizochytrium sp. is characterized by the specified amount of components listed in Table 5, where “Max” refers to the amount of the component that can be present up to the specified amount.

TABLE 5

| | CONCENTRATION (WT/WT) |
|---|---|
| FATTY ACIDS | |
| 14:0 | 6.0%-12.0% |
| 16:0 | 18%-28% |
| 18:0 | MAX 2% |
| 18:1 | MAX 8% |
| 18:2 | MAX 2% |
| 20:4 ARA | MAX 2% |
| 20:5 (N-3) EPA | MAX 3% |
| 22:5 (N-6) DPA | 12%-18% |
| 22:6 (N-3) DHA | MIN 35% |
| OTHERS | MAX 10% |
| ELEMENTAL COMPOSITION | |
| ARSENIC | MAX 0.2 PPM |
| COPPER | MAX 0.05 PPM |
| IRON | MAX 0.2 PPM |
| LEAD | MAX 0.1 PPM |
| MERCURY | MAX 0.04 PPM |
| CHEMICAL CHARACTERISTICS | |
| PEROXIDE VALUE | MAX 5 MEQ/KG |
| FREE FATTY ACID | MAX 0.25% |
| MOISTURE AND VOLATILES | MAX 0.05% |
| UNSAPONIFIABLE MATTER | MAX 4.5% |
| TRANS FATTY ACIDS | MAX 1% |

Compositions useful in the methods herein also include compositions that comprise at least about 90 wt. % of a combination of DPA (n-6) and DHA. In certain embodiments, the compositions may comprise at least about 91, 92, 93, 94, 95, 96, 97, 98, or 99 wt. % of a combination of DPA (n-6) and DHA. In some embodiments, the compositions may comprise at least about 10 wt. % DHA and at least about 10 wt. % DPA (n-6). In other embodiments, the compositions may comprise at least about 15 or 20 wt. % DHA and at least about 15 or 20 wt. % DPA (n-6).

The present invention also provides compositions comprising at least about 90 wt. % of a combination of DPA (n-6) and DHA, and at least one additional fatty acid or a derivative, such as an ester, thereof. In certain embodiments, the compositions may comprise at least about 91, 92, 93, 94, 95, 96, 97, 98, or 99 wt. % of a combination of DPA (n-6) and DHA. In some embodiments, the additional fatty acid may have a boiling point of about 150-170° C. at a pressure of 0.8 mm Hg.

The DHA/DPA (n-6) compositions described above may further comprise less than about 4% of a saturated fatty acid or an ester thereof. In certain embodiments, the compositions may comprise less than about 3.5%, 3.0%, 2.5%, 2.0%, 1.5%, 1.0% or 0.5% of a saturated fatty acid or a derivative thereof.

The DHA in an oil may be in the form of a DHA ester, preferably an alkyl ester, such as a methyl ester, ethyl ester, propyl ester, or combinations thereof, prepared from an algal oil derived from the Thraustochytrid, Schizochytrium sp. An exemplary DHA (ethyl esters) containing oil derived from Schizochytrium sp. is characterized by the specified amount of components listed in Table 4 of WO 2009/006317, incorporated by reference herein. In some of these embodiments, an oil comprises DHA>than about 57% (w/w), particularly >about 70% (w/w) of the total fatty acid content of the oil or unit dose. In some of these embodiments, the ratio of wt % of DHA to wt % of DPAn6 is about 2.5 to about 2.7.

In some embodiments, the composition or oil is characterized by one or more the following fatty acids (or esters thereof, particularly ethyl esters), expressed as wt % of the total fatty acid content. The embodiments provided herein may further comprise about 0.5% or less (w/w) of lauric acid (C12:0). The embodiments provided herein may further comprise about 2% or less (w/w) of myristic acid (C14:0). The embodiments provided herein may further comprise about 0.5% or less (w/w) of myristoleic acid (C14:1). The embodiments provided herein may further comprise about 1% or less of palmitic acid (C16:0). The embodiments provided herein may further comprise about 1% or less (w/w) of linoleic acid (C18:2) (n-6). The embodiments provided herein may further comprise about 3% or less (w/w) of dihomo gamma linolenic acid (C20:3) (n-6). The embodiments provided herein may further comprise about 0.5% or less (w/w) of eicosatrienoic (C20:3) (n-3). The embodiments provided herein may further comprise about 1% or less (w/w) of arachidonic acid (C20:4). The embodiments provided herein may further comprise about 3% or less (w/w) of eicosapentaenoic acid (C20:5) (n-3). The embodiments provided herein may further comprise about 3% or less (w/w) of docosatrienoic acid (22:3). The embodiments provided herein may further comprise about 27% or less (w/w) of decosapentaenoic acid (22:5) (n-6). The embodiments provided herein may further comprise about 10% or less (w/w) of other components. In some of these embodiments, the ratio of wt % of DHA to wt % of DPAn6 is about 2.5 to about 2.7. An oil with the preceding characteristics may comprise ethyl ester oil derived from the oil of Thraustochytrid, Schizochytrium sp.

In some embodiments, another exemplary DHA (free fatty acid) containing oil is characterized by the specified amount of components (as ethyl esters) listed in Table 6, where “Max” refers to the amount of the component that can be present up to the specified amount.

TABLE 6

| FATTY ACIDS | CONCENTRATION (WT/WT) |
|---|---|
| C12:0 | MAX 0.5% |
| C14:0 | MAX 2% |
| C14:1 | MAX 0.5% |
| C16:0 | MAX 1% |
| C18:2 N-6 | MAX 1% |
| C20:3 (N-6) | MAX 3% |
| C20:3 (N-3) | MAX 0.5% |
| C20:4 ARA | MAX 1% |
| C20:5 (N-3) EPA | MAX 3% |
| C22:3 | MAX 3% |
| C22:5 (N-6) DPA | MAX 27% |
| C22:6 (N-3) DHA | MIN 57% |
| % ADDITIONAL COMPONENTS | MAX 8% |

In some embodiments, another exemplary DHA (free fatty acid) containing oil is characterized by the specified amount of components listed in Table 7:

TABLE 7

| | CONCENTRATION (WT/WT) |
|---|---|
| FATTY ACIDS | |
| 10:0 | MAX 0.5% |
| 12:0 | MAX 0.5% |
| 14:0 | MAX 0.5% |
| 14:1 | MAX 0.5% |
| 16:0 | MAX 0.5% |
| 16:1 | MAX 0.5% |
| 18:1 (N-9) | MAX 0.5% |
| 20:5 (N-3) EPA | MAX 0.5% |
| 22:5 (N-3) DPA | MAX 1% |
| 22:6 (N-3) DHA | MIN 95% |
| 28:8 | MAX 1.5% |
| CHEMICAL CHARACTERISTICS | |
| DOCOSAHEXAENOIC ACID | 946 MG/G |
| DOCOSAHEXAENOIC ACID | 98% |
| FREE FATTY ACIDS | 93% |
| TRANS FATTY ACIDS | <1% |

In some embodiments, the present invention further includes use of compositions comprising at least about 70 wt. % DHA and at least about 15, 20, or 25 wt. % DPA (n-6).

In some embodiments, the saturated fatty acid or an ester thereof may contain less than 20 carbons, such as, for example, a saturated fatty acid or an ester thereof that contains 19, 18, 17. 16, 15, 14, 13, 12, 11, 10, 9 or 8 carbons. In certain embodiments, the saturated fatty acid or ester thereof may contain 14 or 16 carbons.

In some embodiments, the composition of DHA may further comprise vitamin E Compounds of the vitamin E group are fat-soluble vitamins with antioxidant properties and include eight related α-, β-, γ-, and δ-tocopherols and the corresponding four tocotrienols. In some embodiments, the vitamin E in the composition is a tocopherol. In some embodiments, the tocopherol is selected from α-, β-, γ-, and δ-tocopherols, or combinations thereof.

In the methods described herein, the composition of DHA is administered to a human subject identified as being negative for the ApoE4 allele. As noted herein, human subjects who do not have the ApoE4 allele appear to derive the most benefit from administration of DHA. In the embodiments herein, the subject is a “subject in need thereof.” A subject in need thereof refers to an individual for whom it is desirable to treat (e.g., a subject diagnosed with AD).

A human subject negative for the ApoE4 allele may be identified by any technique known in the art. Detecting the presence or absence of ApoE4 protein or of DNA encoding such isoform (including the number of alleles, e.g., heterozygous or homozygous, of the relevant ApoE allele) may be carried out either directly or indirectly by any suitable means. A variety of such techniques are known to those skilled in the art. These techniques generally involve the step of collecting a sample of biological material containing either DNA or ApoE from the subject, and then detecting whether the sample contains, and therefore the subject possesses, ApoE4 or DNA encoding the ApoE4 isoform. For example, the detecting step may be carried out by collecting an ApoE sample from the subject (for example, from cerebrospinal fluid, or any other fluid or tissue containing ApoE), and then determining the presence or absence of an ApoE4 isoform in the ApoE sample (e.g., by-isoelectric focusing or immunoassay using allele specific anti-ApoE antibodies) Immunochemical methods include those described in WO94/09155 “Methods of Detecting Alzheimer's Disease” and U.S. Pat. No. 5,508,167, “Methods of screening for Alzheimer's disease” by Roses et al., which discloses methods for detecting the presence or absence of ApoE4 for the diagnosis of AD. In the alternative, the detecting step may be carried out by collecting a biological sample containing DNA from the subject, and then determining the presence or absence of DNA encoding an ApoE4 isoform in the biological sample. Any biological sample which contains the DNA of that subject may be employed, including tissue samples and blood samples, with blood cells being a particularly convenient source. Numerous techniques for detecting the presence of one or two ApoE4 alleles in a subject are known, including but not limited to those described in U.S. Pat. No. 5,508,167 “Methods of screening for Alzheimer's disease” by Roses et al.; U.S. Pat. No. 5,773,220 “Determination of Alzheimer's disease risk using apolipoprotein E and α-1 Antichymotrypsin Genotype Analysis” by DeKosy and Kamboh; and U.S. Pat. No. 5,935,781 “Apolipoprotein E polymorphism and treatment of Alzheimer's disease” by Poirier. These systems include mass-spectrometry-based procedures such as matrix-assisted laser desorption/ionization, denaturing high pressure liquid chromatography, oligonucleotide ligation assays, and solid-phase-array-type systems. Most of these approaches include some form of enzymatic DNA amplification such as polymerase chain reaction (PCR), ligase chain reaction, or rolling-circle amplification. In some embodiments, the method of determining apoE genotype can use PCR-based methods-primarily PCR of a portion of the apoE gene followed by digestion with restriction enzymes that recognize the DNA substitutions that distinguish the alleles and gel electrophoresis or most currently, using TaqMan real time PCR, a fluorescence detection system that relies upon a 5′-nuclease assay with allele specific fluorogenic probes. These TaqMan probes only fluoresce when they are bound to the template. This method is described in Macleod et al., 2001, Eur J Clinical Investigation 31(7):570-3. Commercial products for determining apoE genotype are available from LabCorp and Athena Diagnostics. Other methods, such as the Invader assay by Third Wave Technologies Inc. (Madison, Wis.), that amplify the generated signal probe rather than the target DNA can also be used.

An enzyme-free approach capable of analyzing single-nucleotide variations directly from human genomic DNA is available from Nanosphere Inc. (Northbrook, Ill.). The gold-nanoparticle-based assay relies on two consecutive hybridization steps. First, genomic DNA is hybridized to allele-specific microarray-bound oligonucleotides. Next, DNA-modified gold nanoparticles hybridize to a sequence in close vicinity to the ApoE4 allele. Finally, a signal-amplification step is performed during which elementary silver is deposited on the gold nanoparticles and the light scattering induced by an evanescent wave in the glass substrate is measured and quantified.

The human subject for treatment may be selected for treatment by the methods of the present invention based upon knowledge of the ApoE4 profile of that individual patient (i.e., the absence of ApoE4 allele). The ApoE profile may be obtained in the manner described above. It is not necessary that such screening or profiling be at the same time or place, or by the same individual, as the individual making the selection for therapy, so long as the selection is based upon this information.

In some embodiments, where the method includes the step of identifying a human subject negative for the ApoE4 allele, the method may comprise a prior step of testing a human subject for the presence or absence of the ApoE4 allele. Subjects negative for the ApoE4 allele may be positive for the ApoE2 or ApoE3 allele. In some embodiments, the subject to be treated is heterozygous for the ApoE2 or ApoE3 allele. In some embodiments, the subject to be treated is homozygous for the ApoE2 or ApoE3 allele.

In some embodiments, the human subject identified as being negative for the ApoE4 allele may or may not be diagnosed with an age-related cognitive disorder, dementia, or AD. As further described below, the composition of DHA may be administered to a patient who is healthy or who has been diagnosed with an age-related cognitive disorder but not dementia or AD, to prevent or lower the risk of developing dementia or AD. In some embodiments, the human subject identified as being negative for the ApoE4 allele is diagnosed with an age-related cognitive disorder, dementia, or AD. As such, the DHA composition may be administered to a human subject in need thereof an amount effective (either alone or in combination with another anti-Alzheimer's therapy) to reduce the severity or slow the progression of an age-related cognitive disorder, dementia, or AD. Any number of techniques may be used to diagnose whether a human subject is afflicted with AD. As used herein “diagnose,” “diagnosis,” and “diagnosing” and variants thereof are used interchangeably herein to refer to the identification of a disease or condition on the basis of its signs and symptoms. A “positive diagnosis” indicates that the disease or condition, e.g., an age-related cognitive disorder, dementia, or AD, or a potential for developing the disease or condition, has been identified. In contrast, a “negative diagnosis” indicates that the disease or condition, or a potential for developing the disease or condition, has not been identified. In the case of a positive diagnosis, an individual may be prescribed treatment to reverse, decrease or eliminate the signs of an age-related cognitive disorder, dementia, or AD, including the use of a DHA composition of the invention.

While different tests are available and have been applied to assessing the presence and stage of an age-related cognitive disorder, dementia, or AD, the tests and criteria for diagnosing and staging of these disorders can use those promulgated in the World Health Organization International Classification of Diseases ICD-10 and/or Diagnostic and Statistical Manual for Mental Disorders, Fourth Edition (DSM-IV), as discussed herein. The ICD is the international standard diagnostic classification for epidemiological and clinical use while the DSM is published by the American Psychiatric Association and provides diagnostic criteria for mental disorders. For instance, diagnosis of dementia and AD is described specifically in the ICD-10 Classification of Mental and Behavioral Disorders.

In some embodiments, various cognitive and psychological tests are well-known in the art and may be implemented in the methods described herein. These tests include, among others, the Mini-Mental State Examination (MMSE), Cambridge Neuropsychological Test Automated Battery (CANTAB), Alzheimer's Disease Assessment Scale-cognitive test (ADAS-cog), Wisconsin Card Sorting Test, Verbal and Figural Fluency Test and Trail Making Test. In particular, ADAS-cog may be used for diagnosing as well as assessing the effectiveness of therapy. Furthermore, a combination of any of the foregoing tests may be used.

In some embodiments, the diagnostic technique may include brain imaging techniques, including, among others, electroencephalography (EEG), magnetoencephlography (MEG), Positron Emission Tomography (PET), Single Photon Emission Computed Tomography (SPECT), Magnetic Resonance Imaging (MRI), functional Magnetic Resonance Imaging (fMRI), computerized tomography, and long-term potentiation. Furthermore, a combination of any of the foregoing diagnostic techniques may be used.

EEG measures electrical activity of the brain and is typically accomplished by placing electrodes on the scalp at various landmarks and recording greatly amplified brain signals. MEG, which is allied with EEG, measures the magnetic fields that are linked to electrical fields. MEG is used to measure spontaneous brain activity, including synchronous waves in the nervous system.

PET provides a measure of oxygen utilization and glucose metabolism. In this technique, a radioactive positron-emitting tracer is administered, and tracer uptake by the brain is correlated with brain activity. These tracers emit gamma rays which are detected by sensors surrounding the head, resulting in a three-dimensional map of brain activation. As soon as the tracer is taken up by the brain, the detected radioactivity occurs as a function of regional cerebral blood flow (“CBF”) and during activation, an increase in CBF and neuronal glucose metabolism can be detected. Use of PET imaging for diagnosis is described in, for example, Noble and Scarmeas, 2009, Int. Rev. Neurobiol. 84C:133-149, incorporated herein by reference.

MRI and fMRI capitalize on the fact that one property of atomic nuclei, their spins, can be manipulated by exposing them to a large magnetic force. While the subject lies with his/her head in a powerful magnet (1.5 to 5 Teslas in force), a short-wave radio wave antenna varies the magnetic field in a way that is much weaker than the main magnet. The varying pulse produces a resonance signal from the nuclei that can be quantified in 3D and digitized.

In some embodiments, the diagnostic technique may be based on measuring the relative levels of two biochemical markers associated with AD, namely Tau and β-amyloid (A beta 42), in cerebrospinal fluid (CSF) of human subjects (see, e.g., de Jong et al., 2006, The Journals of Gerontology Series A: Biological Sciences and Medical Sciences 61:755-758; Shaw et al., 2009, Ann. Neurol. 65(4):403-13). Levels of Tau, Aβ42, and p-tau181 in CSF can be measured by enzyme-linked immunosorbent assays using antibodies directed against the biochemical markers.

As will be understood by those skilled in the art, these diagnostic techniques used for assessing whether a human subject has an age-related cognitive disorder, dementia, or AD may also be used to assess the effectiveness of administering DHA in treating or preventing an age-related cognitive disorder, dementia, or AD. In particular, non-invasive cognitive tests, such as the ADAS-cog test, may be used for assessing the effectiveness of the treatment, as noted in the Example.

For treating or preventing an age-related cognitive disorder, dementia, or AD, the compositions of DHA are administered in an amount effective, either alone or in combination with another anti-Alzheimer's therapy, to treat or prevent an age-related cognitive disorder, dementia, or AD. The terms “treat”, “treatment” and “treating” are used interchangeably herein to refer to therapeutic treatment and prophylactic or preventative measures, wherein the object is to treat, prevent or slow the progression of an undesired physiological condition, disorder or disease, or obtain beneficial or desired clinical results. For purposes herein, beneficial or desired clinical results include, but are not limited to, alleviation of symptoms associated with an age-related cognitive disorder, dementia, or AD; diminishment of the extent of the condition associated with an age-related cognitive disorder, dementia, or AD; stabilization (i.e., not worsening) of the state of the condition, disorder or disease associated with an age-related cognitive disorder, dementia, or AD; delay in onset or slowing of the condition, disorder or disease progression associated with an age-related cognitive disorder, dementia, or AD; amelioration of the condition, disorder or disease state, remission (whether partial or total) the condition, disorder or disease associated with an age-related cognitive disorder, dementia, or AD, whether detectable or undetectable; or enhancement or improvement of the condition, disorder or disease assorted with an age-related cognitive disorder, dementia, or AD. Treatment includes eliciting a clinically significant response, without excessive levels of side effects. Treatment also includes prolonging survival as compared to expected survival if not receiving treatment.

In some embodiments, the DHA compositions are administered in an amount effective to raise the DHA levels in the subject sufficient to treat the an age-related cognitive disorder, dementia, or AD. In some embodiments, the human subject in need thereof and identified as being negative for the ApoE4 allele is administered an oral dosage formulation comprising DHA in an amount sufficient to raise the plasma phospholipid DHA levels at about least 3 fold in six months. In some embodiments, the formulation is provided in the substantial absence of EPA.

In some embodiments, the human subject in need thereof and identified as being negative for the ApoE4 allele is administered an oral dosage formulation comprising DHA in an amount sufficient to raise the cerebrospinal fluid DHA levels by at least 30%. In some embodiments, the formulation is provided in the substantial absence of EPA.

In the course of examination of a subject, a medical professional can determine that administration of DHA pursuant to one of the methods described herein is appropriate for the subject, or the physician can determine that the subject's condition can be improved by the administration of DHA pursuant to one of the methods described herein. Prior to prescribing any DHA regimen, the physician can counsel the subject, for example, on the various risks and benefits associated with the regimen. The subject can be provided full disclosure of all the known and suspected risks associated with the regimen. Such counseling can be provided verbally, as well as in written form. In some embodiments, the physician can provide the subject with literature materials on the regimen, such as product information, educational materials, and the like.

The present invention is also directed to methods of educating consumers about the methods of treating neurological disorders, the method comprising distributing the DHA dosage forms with consumer information at a point of sale. In some embodiments, the distribution will occur at a point of sale having a pharmacist or healthcare provider.

The term “consumer information” can include, but is not limited to, an English language text, non-English language text, visual image, chart, telephone recording, website, and access to a live customer service representative. In some embodiments, consumer information will provide directions for use of the DHA unit dosages according to the methods described herein, appropriate age, use, indication, contraindications, appropriate dosing, warnings, telephone number, and website address. In some embodiments, the method further comprises providing professional information to relevant persons in a position to answer consumer questions regarding use of the disclosed regimens according to the methods described herein. The term “professional information” includes, but is not limited to, information concerning the regimen when administered according to the methods of the present invention that is designed to enable a medical professional to answer customer questions.

A “medical professional,” includes, for example, a physician, physician assistant, nurse practitioner, pharmacist and customer service representative. All of the various aspects, embodiments and options described herein can be combined in any and all variations.

In some embodiments, the DHA is administered in a single dosage form, i.e., a dosage form, or in two or more dosage forms. As used herein, “dosage form” refers to the physical form for the route of administration. The term “dosage form” can refer to any traditionally used or medically accepted administrative forms, such as oral administrative forms, intravenous administrative forms, or intraperitoneal administrative forms. In some embodiments, the DHA is administered in a single dose, i.e., a unit dose. As used herein, a “unit dose” refers to an amount of DHA administered to a subject in a single dose, e.g., in a gel capsule. The term “unit dose” can also refer to a single unit of pharmaceutically suitable solid, liquid, syrup, beverage, or food item, that is administered within a short period of time, e.g., within about 1 minute, 2 minutes, 3 minutes, 5 minutes, 10 minutes, 20 minutes, or 30 minutes.

In some embodiments, the subject to be treated can be administered at least one unit dose per day. In some embodiments, the dosage forms can be taken in a single application or multiple applications per day. For example, if four capsules are taken daily, each capsule comprising about 500 mg DHA, then all four capsules could be taken once daily, or 2 capsules could be taken twice daily, or 1 capsule could be taken every 6 hours. Various amounts of DHA can be in a unit dose. In some embodiments, the unit dose comprises about 430 mg, about 450 mg, about 500 mg, about 550 mg, about 600 mg, about 650 mg, about 700 mg, about 750 mg, about 800 mg, about 850 mg, about 900 mg, about 950 mg, about 1 g, or about 1.5 g, DHA.

In some embodiments, the dosage form has a total weight of about 0.2 g to about 2 g. By way of example and not limitation, a capsule can contain a total weight an algal oil of about 0.2 g, where the algal oil contain comprises DHA at a certain wt % of the total fatty acid content of the algal oil. In some embodiments, the dosage form has a total weight of about 0.2 g, about 0.25, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g or about 1.05 g.

For the purposes herein, the composition of DHA may be administered daily and for a time period sufficient to provide a therapeutic benefit to the subject. As used herein, “daily dosage,” “daily dosage level,” “daily dosage amount” or “per day dosage” refer to the total amount of DHA (e.g., in the form of free fatty acids, alkyl esters, or triglycerides) administered per day (about 24 hour period). For example, administration of DHA to a subject at a dosage of 2 g per day means that the subject receives a total of 2 g of DHA on a daily basis, whether the DHA is administered as a single dosage form comprising 2 g DHA, or alternatively, four dosage forms comprising 500 mg DHA each (for a total of 2 g DHA). The composition of DHA may be taken in a single application or multiple applications per day. For example, if four capsules are taken daily, each capsule comprising 500 mg DHA, then all four capsules could be taken once daily, or 2 capsules could be taken twice daily, or 1 capsule could be taken every 6 hours. In some embodiments, the daily amount of DHA is administered at least once per day (e.g., single dosage form daily) or at least twice per day (e.g., in two or more dosage forms daily). In some embodiments, the DHA is administered at least two times weekly.

In some embodiments, the DHA is administered in an amount of from about 1.5 mg per kg body weight per day to about 125 mg per kg body weight per day. In some embodiments, the DHA is administered in an amount of from about 150 mg to about 10 g per day; from about 0.5 g per day to about 5 g per day; or from about 1 g per day to about 5 g per day.

In some embodiments, the daily amount of DHA administered comprises about 200 mg, 400 mg, 450 mg, 500 mg, 520 mg, 540 mg, 600 mg, 700 mg, 800 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA per day. In some embodiments, the DHA is administered in an amount of at least about 1 g per day.

In some embodiments, the daily dose of DHA administered to a human subject ranges from about 860 mg up to about 6 grams, particularly from about 1.7 grams up to about 6 grams, from about 2.6 grams up to about 6 grams, particularly from about 3.4 grams up to about 6 grams, particularly from about 4.3 grams to about 6 grams and more particularly from about 5.1 grams to about 6 gram. In some embodiments the daily dose of DHA administered to a human subject ranges from about 860 mg up to about 4 grams, particularly from about 1.7 grams up to about 4 grams, from about 2.6 grams up to about 4 grams, and more particularly from about 3.4 grams up to about 4 grams. In some embodiment the daily dose of DHA administered to a human subject ranges from about 860 mg up to about 1 gram, particularly from about 860 mg up to about 950 mg. In some embodiments, the daily dose of DHA administered ranges from about 1.7 grams up to about 2 grams, particularly from about 1.7 gram up to about 1.8 grams. In some embodiments, the daily dose of DHA administered to a human subject ranges from about 2.6 grams up to about 3 grams, particularly from about 2.6 grams up to about 2.8 grams. In some embodiments, the daily dose of DHA administered to a human subject is from about 3.4 grams up to about 4 grams, particularly from about 3.4 grams up to about 3.8 grams. In some embodiments, the daily dose of DHA administered to a human subject is from about 4.3 to about 5 grams, particularly from 4.3 grams to about 4.8 grams. In some embodiments, the daily dose of DHA administered to a human subject is from about 5.1 to about 6 grams, particularly from about 5.1 to about 5.7 grams.

In some embodiments, the daily dose is provided as a unit dose.

Various amounts of DHA may be in a dosage form. In some embodiments, the dosage form comprises less than about 5 g of DHA, about 100 mg to about 3.8 g DHA, about 200 mg to about 3.6 g of DHA, about 500 mg to about 4.0 g DHA, or about 1 g to about 2.0 g DHA. In some embodiments, the dosage form comprises less than about 4 g of DHA, about 200 mg to about 3.9 g DHA, about 500 mg to about 3.7 g of DHA, about 750 mg to about 3.5 g DHA, or about 1 g to about 2 g DHA. In some embodiments, the dosage form of DHA is less than about 3.8 g DHA, about 900 mg to about 3.6 g DHA, or about 1.8 g to about 2.7 g of DHA. In some embodiments, the dosage form of DHA comprises about 200 mg, 400 mg, 450 mg, 500 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA.

Administration of the DHA may be achieved using various regimens. For example, in some embodiments, administration of the DHA is daily on consecutive days, or alternatively, the dosage form is administered every other day (bi-daily). Administration may occur on one or more days. For example, in some embodiments the DHA is administered daily for the duration of the subject's lifetime, or from 1 year to 20 years or 5 years to 10 years. In some embodiments, administration of the DHA dosage form occurs for 7, 14, 21, or 28 days. In some embodiments, the DHA is administered for at least 6 months, for at least 1 yr, for at least 1.5 yrs., for at least 2 yrs., or for at least 5 yrs. In some embodiments, administration of the DHA occurs until a symptom of dementia or AD, e.g., loss of cognitive ability, is halted or reduced, the target being determined by a medical professional.

In some embodiments, the DHA is administered continuously. The term “continuous” or “consecutive,” as used herein in reference to “administration,” means that the frequency of administration is at least once daily. Note, however, that the frequency of administration can be greater than once daily and still be “continuous” or “consecutive,” e.g., twice or even three or four times daily, as long as the dosage levels as specified herein are achieved.

The term “administering” or “administration” of the composition refers to the application of the composition, e.g., oral or parenteral (e.g., transmucosal, intravenous, intramuscular, subcutaneous, rectal, intravaginal, or via inhalation) to the subject. Administering would also include the act of prescribing a composition described herein to a subject by a medical professional for treatment of AD. Administering can also include the act of labeling a composition, i.e., instructing a subject to administer a composition, in a manner as provided herein for treatment of AD. By way of example, administration may be by parenteral, subcutaneous, intravenous (bolus or infusion), intramuscular, or intraperitoneal routes. Dosage forms for these modes of administration may include conventional forms, either as liquid solutions or suspensions, solid forms suitable for solution or suspension in liquid prior to injection, or as emulsions.

Although fatty acids such as DHA can be administered topically or as an injectable, a preferred route of administration is oral administration. Preferably, the DHA composition is administered to individuals in the form of nutritional supplements, foods, pharmaceutical formulations, or beverages, particularly foods, beverages, or nutritional supplements, more particularly, foods and beverages, more particularly foods. A preferred type of food is a medical food (e.g., a food which is in a formulation to be consumed or administered externally under the supervision of a physician and which is intended for the specific dietary management of a disease or condition for which distinctive nutritional requirements, based on recognized scientific principles, are established by medical evaluation.).

In some embodiments, the dosage form is a pharmaceutical dosage form. “Pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the compounds (e.g., DHA), compositions, and dosage forms of the present invention are pharmaceutically acceptable.

The DHA can be formulated in a dosage form. These dosage forms can include, but are not limited to, tablets, capsules, cachets, pellets, pills, gelatin capsules, powders, and granules; and parenteral dosage forms which include, but are not limited to, solutions, suspensions, emulsions, coated particles, and dry powder comprising an effective amount of the DHA as taught in this invention. In some embodiments, the dosage form can be inserted or mixed into a food substance. Various substances are known in the art to coat particles, including cellulose derivatives, e.g., microcrystalline cellulose, methyl cellulose, carboxymethyl cellulose; polyalkylene glycol derivatives, e.g., polyethylene glycol; talc, starch, methacrylates, etc. In some embodiments, the dosage form is a capsule, wherein the capsule is filled with a solution, suspension, or emulsion comprising the DHA. It is also known in the art that the active ingredients can be contained in such formulations with pharmaceutically acceptable excipients such as diluents, fillers, disintegrants, binders, lubricants, surfactants, hydrophobic vehicles, water soluble vehicles, emulsifiers, buffers, humectants, moisturizers, solubilizers, preservatives, flavorants, taste-masking agents, sweeteners, and the like. Suitable excipients can include, e.g., vegetable oils (e.g., corn, soy, safflower, sunflower, or canola oil). In some embodiments, the preservative can be an antioxidant, e.g., sodium sulfite, potassium sulfite, metabisulfite, bisulfites, thiosulfates, thioglycerol, thiosorbitol, cysteine hydrochloride, α-tocopherol, and combinations thereof. The means and methods for administration are known in the art and an artisan can refer to various pharmacologic references for guidance. For example, “Modern Pharmaceutics,” Banker & Rhodes, Informa Healthcare, 4th ed. (2002); “Goodman & Gilman's The Pharmaceutical Basis of Therapeutics,” McGraw-Hill, New York, 10th ed. (2001); and Remingtons's Pharmaceutical Sciences, 20th Ed., 2001 can be consulted.

The DHA of the present invention is orally active and this route of administration can be used for the methods described herein. Accordingly, administration forms can include, but are not limited to, tablets, dragees, capsules, caplets, gelatin capsules, and pills, which contain the DHA and one or more suitable pharmaceutically acceptable carriers.

For oral administration, the DHA can be administered as an oil or it can be formulated readily by combining it with a pharmaceutically acceptable carrier or with pharmaceutically acceptable carriers. Pharmaceutical acceptable carriers are well known in the art. Such carriers enable the compounds of the invention to be formulated as tablets, gelatin capsules, pills, dragees, capsules, liquids, gels, syrups, slurries, suspensions and the like, for oral ingestion by a subject to be treated. In some embodiments, the dosage form is a tablet, gelatin capsule, pill or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, vegetable oil (e.g., soybean oil), and polyvinylpyrrolidone (PVP). If desired, disintegrating agents can be added, such as, but not limited to, the cross-linked polyvinyl pyrrolidone, agar, or alginic acid or a salt thereof such as sodium alginate. Pharmaceutical preparations which can be used orally include, but are not limited to, push-fit capsules made of gelatin, as well as soft, sealed capsules made of gelatin and a plasticizer, such as glycerol or sorbitol. Capsule shells can be composed of non-animal derived ingredients, i.e., vegetarian ingredients, such as carrageenan, alginate, modified forms of starch, cellulose and/or other polysaccharides. In specific embodiments, the gelatin capsules may be porcine, bovine, vegetarian, or alginate gelatin capsules. All formulations for oral administration should be in dosages suitable for such administration.

It should be understood that in addition to the ingredients particularly mentioned above, the formulations of this invention can include other suitable agents such as flavoring agents, preservatives, and antioxidants. In particular, it is desirable to mix the microbial oils with an antioxidant to prevent oxidation of the DHA. Such antioxidants are pharmaceutically acceptable and can include vitamin E, carotene, BHT or other antioxidants known to those of skill in the art.

In some embodiments, the dosage form is a nutraceutical dosage form. The term “nutraceutical” refers to any substance that is (1) a sole item of a meal or diet that provides medical and/or health benefits, or (2) a product that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these ingredients that provides medical and/or health benefits. The medical and/or health benefits can include reducing the risk of a neurological disorder described herein.

In some embodiments, the DHA can be provided in a dietary supplement, medical food or animal feed. “Dietary supplement” refers to a compound or composition used to supplement the diet of an animal or human. In some embodiments, the dietary supplement can further comprise various “dietary ingredients” intended to supplement the diet. “Dietary ingredients” can further include: vitamins, minerals, herbs or other botanicals, amino acids, and substances such as enzymes, organ tissues, glandulars, and metabolites. Dietary ingredients can also include extracts or concentrates. In some embodiments, the dosage form of DHA is administered in a dietary supplement.

In some embodiments, the DHA is provided as a medical food for the dietary management of DHA levels in a human subject who is suffering from Alzheimer's disease, particularly one suffering mild to moderate AD. In some embodiments, the DHA is provided in an amount sufficient to increase the DHA levels in plasma phospholipid DHA of a subject who is ApoE4 negative and who is suffering from AD, particularly suffering from mild to moderate AD, more particularly from mild AD.

In some embodiments, DHA is provided as a medical food in an amount sufficient to increase the DHA levels in cerebrospinal fluid of a human subject suffering from AD, particularly mild to moderate AD, more particularly with mild AD.

The present invention is also directed to use of an oral dosage form consisting essentially of about 430 mg to about 6 g of docosahexaenoic acid (DHA) wherein the dosage form comprises less than about 1% eicosapentaenoic acid (EPA) and less than about 2% docosapentaenoic acid 22:5n-6 (DPAn6). In some embodiments, the oral dosage form is a unit dosage form, in particular, a gelatin capsule. Optionally the gelatin capsule also comprises a colorant, flavoring, and/or antioxidant.

The present invention is also directed to use of oral dosage forms comprising: (a) about 200 mg to about 4 g of DHA, wherein the DHA is about 40% to about 99.5% (w/w) or more of the total fatty acid content of the dosage form; and (b) a pharmaceutically acceptable excipient, wherein the dosage form is substantially free of EPA, and wherein the DHA, such as a DHA alkyl ester, is derived from an algal source.

The present invention includes gelatin capsules that are hard or soft gelatin capsules. In some embodiments, the encapsulating material comprises a gelatin, a plasticizer, and water. In certain embodiments, the encapsulating material is vegetarian, i.e., made from non-animal derived material, including plants, seaweed (for example, carrageenan), food starch, modified corn starch, potato starch, and tapioca. In other embodiments, the encapsulating material is derived from animals, including porcine, bovine, and fish-based materials, such as gelatins. Plasticizers of the invention include glycerin, glycerol, polyols, and mixtures thereof. In some embodiments, the plasticizer is a high boiling point polyol, such as glycerol or sorbitol.

In some embodiments, the gelatin capsule is a soft-gelatin capsule made from gelatin, glycerol, and water, and filled with DHA and an antioxidant. In certain embodiments, the gelatin capsule is animal or vegetable derived. In some embodiments, the gelatin capsule comprises a 0.5 gram dosage form, wherein the fill weight of the weight of the dosage form is from about 450 mg to about 550 mg, and wherein the gelatin capsule comprises from about 430 mg to about 480 mg DHA. In some embodiments, the gelatin capsule comprises about 450 mg DHA per 500 mg of the dosage form. In some embodiments, the gelatin capsule comprises about 450 mg DHA per 500 mg of the dosage form. In some embodiments, the gelatin capsule comprises a 1 gram dosage form, wherein the fill weight of the dosage form is from about 950 mg to about 1050 mg, and wherein the gelatin capsule comprises from about 860 mg to about 950 mg DHA per 1000 mg of the dosage form. In some embodiments, the gelatin capsule comprises about 900 mg DHA per 1,000 g of the dosage form.

In certain embodiments, the gelatin capsule is vegetarian. In some embodiments, the capsule preparation comprises no animal products, and comprises glycerol (and/or other polyols), seaweed extract (carrageenan) and water. In some embodiments, the water is purified. In some embodiments, color, flavor and/or sweeteners are added. During encapsulation, in some embodiments, fractionated coconut oil is used as a lubricant.

In some embodiments, the gelatin capsule comprises a capsule preparation, an active, and optionally a colorant and/or antioxidant. In another embodiment i) the capsule preparation comprises gelatin (bovine acid hide), glycerin, and purified water, ii) the active comprises DHA-EE, iii) the optional colorant is selected from titanium dioxide, FD&C Yellow #5, FD&C Red 40, and mixtures thereof; and iv) the antioxidant is ascorbyl palmitate. In some embodiments, the raw materials are USP raw materials.

In some embodiments, the gelatin capsules are soft gelatin capsules of about 1 g, having the specifications within the limits set forth in Table 8:

TABLE 8
Specifications for 1 gram DHA Ethyl Ester Gelatin Capsules

| TEST | SPECIFICATION |
|---|---|
| DHA EE CONTENT, PER CAPSULE | 855-945 MG |
| AVERAGE FILL WEIGHT | 950-1050 MG |
| DISINTEGRATION | COMPLIES USP |
| ACID VALUE | MAX 2 MG KOH/G |
| PEROXIDE VALUE (PV) | MAX 10 MEQ/KG |
| ANISIDINE VALUE (AV) | MAX 20 |
| MICROBIAL LIMITS TESTS | COMPLIES WITH <61> USP |

Set forth in Table 9 is a list of components that are, in some embodiments, used in the manufacture of a DHA-EE soft gelatin capsule, and at least one corresponding function for each component.

TABLE 9
List of Components in 1 gram DHA
Ethyl Ester Soft Gelatin Capsules

| COMPONENT | FUNCTION |
|---|---|
| 900 MG DHA EE | ACTIVE |
| GELATIN, BOVINE ACID HIDE | CAPSULE PREPARATION |
| GLYCERIN | CAPSULE PREPARATION |
| PURIFIED WATER | CAPSULE PREPARATION |
| TITANIUM DIOXIDE | COLORANT |
| FD&C YELLOW #5 | COLORANT |
| FD&C RED #40 | COLORANT |

The present invention is also directed to kits or packages comprising one or more dosage forms to be administered according to the methods described herein. A kit or package can contain one dosage form, or more than one dosage form (i.e., multiple dosage forms). If multiple dosage forms are present in the kit or package, the multiple dosage forms can be optionally arranged for sequential administration. The kits can contain dosage forms of a sufficient number to provide convenient administration to a subject who has a chronic condition and requires long-term administration of the DHA of the present invention. For example, in some embodiments, the kit provides dosage forms of a sufficient number for 1, 2, 3 or 4 months of daily administration of the DHA. In some embodiments of the present invention, the kit comprises dosage forms for shorter periods of administration, e.g., the kit can contain about 7, 14, 21, 28 or more dosage forms for oral administration, each dosage form comprising about 450 mg to about 12.05 g DHA and intended for ingestion on successive days.

The kits can optionally contain instructions associated with the dosage forms of the kits. Such instructions can be in a form prescribed by a governmental agency regulating the manufacture, use or sale of pharmaceutical products, which notice reflects approval by the agency of the manufacture, use or sale for human administration to treat a condition or disorder. The instructions can be in any form which conveys information on the use of the dosage forms in the kit according to the methods described herein. By way of example and not limitation, the instructions can be in the form of printed matter, or in the form of a pre-recorded media device.

In some embodiments, the methods described herein can also be used in combination with other therapies, including pharmaceutical products, to treat or prevent AD. Thus in some embodiments, the DHA is administered adjunctively with another anti-Alzheimer's therapy. As used herein, an “anti-Alzheimer's therapy” refers to any therapy including therapeutic compounds and compositions that can be used for treating or preventing AD in a human subject. The anti-Alzheimer's therapy may be administered sequentially. In such embodiments, the DHA may be administered subsequent to or prior to administration of the anti-Alzheimer's therapy. In some embodiments, the interval between administration of DHA and the anti-Alzheimer's therapy can be minutes, hours, or days, as appropriate for the treatment and effectiveness of the combination treatment. In some embodiments, the anti-Alzheimer's therapy may be administered simultaneously. In such embodiments, the DHA and anti-Alzheimer's therapy if administered in the form of compositions, may be administered in a single composition or separately as independent compositions.

Whether the anti-Alzheimer's therapy is administered sequentially or simultaneously, the DHA and the anti-Alzheimer's therapy may be administered by the same route and manner of administration or by different routes of administration. For example, the DHA and the anti-Alzheimer's drug may be administered orally while in some embodiments, the DHA may be administered orally and the anti-Alzheimer's drug may be administered parenterally.

In some embodiments, the anti-Alzheimer's drug administered adjunctively with DHA is an acetylcholinesterase inhibitor. As used herein, an “acetylcholinesterase inhibitor” refers to any compound or composition that inhibits or reduces the activity of acetylcholinesterase. Suitable acetylcholinesterase inhibitors include, by way of example and not limitation, tacrine, donepezil, rivastigmine, and galantamine.

In some embodiments, the anti-Alzheimer's drug administered adjunctively with DHA comprises a NMDA receptor antagonist. NMDA receptor antagonists are a class of compounds or compositions that work to antagonize, or inhibit the action of, the N-methyl d-aspartate receptor (NMDAR), i.e., receptors that are characterized by binding of n-methyl d-aspartate. In some embodiments, a suitable NMDA receptor antagonist useful for treating AD is memantine.

In some embodiments, the anti-Alzheimer's drug administered adjunctively with DHA comprises Selegeline, Ginkgo biloba, B complex vitamins, calcium channel blockers, HGM CoA reductase inhibitors (including statins), policosanols, fibrates, Clioquinol, and other natural products (e.g., curcumin, lignans, phytoestrogens, phytosterols, niacin, and vitamin supplements).

In some embodiments, the anti-Alzheimer's drug administered adjunctively with DHA is an AD vaccine. In some embodiments, the vaccine is a vaccine against β-amyloid protein. The vaccine may be modified or synthetic forms of β-amyloid that can elicit an immune response against the endogenous β-amyloid protein of the human subject. Vaccines using synthetic peptides of β-amyloid have been used to increase the rate of clearance of abnormal β-amyloid from human subject affected by AD. In some embodiments, a vaccine also includes passive immunization by administering antibodies produced against β-amyloid protein. These antibodies may be polyclonal, monoclonal, non-human, humanized, or human, as is understood in the art. In some embodiments, the passive immunization is based on a humanized monoclonal antibody against the β-amyloid protein. The antibody may be administered in a suitable manner, in particular by parenteral administration.

In some embodiments, the anti-Alzheimer's drug administered adjunctively with DHA is a secretase inhibitor. Two types of secretases, β- and γ-, are known to act on the amyloid precursor protein (APP) to cleave the protein into fragments. Sequential cleavage by β-secretase (BACE) and γ-secretase produces the amyloid-β peptide fragment that aggregates into plaques in the brains of Alzheimer's disease patients. Various β- and γ-secretase inhibitors that are described in the art and can be administered with DHA include, among others, those described in U.S. Pat. No. 6,756,511 “Gamma-secretase inhibitors” by Castro Pineiro et al. (γ-inhibitor), U.S. Pat. No. 7,049,296 “Gamma-secretase inhibitors” by Castro Pineiro et al. (γ-inhibitor), U.S. Pat. No. 7,435,748 “Gamma-secretase inhibitors” by Castro Pineiro et al. (γ-inhibitor), U.S. Pat. No. 7,452,899 “Gamma-secretase inhibitors” by Bettati et al. (γ-inhibitor), U.S. Pat. No. 6,753,163 “Alzheimer's disease secretase, APP substrates therefor, and uses therefor” by Gurney et al. (β-inhibitor), U.S. Pat. No. 7,291,620 “N-alkyl phenylcarboxamide beta-secretase inhibitors for the treatment of Alzheimer's disease” by Coburn et al. (β-inhibitor); U.S. Pat. No. 7,390,925 “Oxime-containing acyl guanidines as beta-secretase inhibitors” by Wu et al. (β-inhibitor), and US Patent Publication No. 2009/0111832 “Imidazolidinone Compounds Useful as Beta-Secretase Inhibitors for the Treatment of Alzheimer's Disease” Barrow et al. (β-inhibitor). All references are incorporated herein by reference.

In some embodiments, other non-DHA compounds and compositions having therapeutic effect on AD may be administered adjunctively with DHA. As noted above, in some embodiments, the composition of DHA may be administered adjunctively with an anti-inflammatory agent. In some embodiments, these anti-inflammatory agents, include non-steroidal anti-inflammatory drugs (NSAID), e.g., aspirin, ibuprofen, naproxen, celecoxib, ketoprofen, piroxicam, and sulindac; steroidal anti-inflammatory agents, e.g., glucocorticosteroid and prednisone; and herbal type anti-inflammatory agents, e.g., ginkgo biloba and tumeric.

In some embodiments, the composition of DHA is administered adjunctively with compounds that affect cholesterol metabolism, particularly a cholesterol lowering agent. These include among others, bile acid binding resins, e.g., cholestyramine and cholestipol; fibric acid derivatives, e.g., gemfibozil and clofibrate; and a HMG CoA reductase inhibitor, for example statin compounds, such as lovastatin, rosuvastatin, pravastatin, atorvastatin and simvastatin.

In some embodiments, the composition of DHA is administered adjunctively with an anti-oxidant, including, among others, vitamin E, e.g., α-, β-, γ- and δ tocopherols; resveratol; vitamin C; acetyl-L-carnitine, and α-lipoic acid.

In some embodiments, the composition of DHA is administered adjunctively with peroxisome proliferation receptor-gamma (PPAR gamma) agonists. Peroxisome proliferator-activated receptor gamma (PPAR-gamma or PPARG), also known as the glitazone receptor, or NR1C3 (nuclear receptor subfamily 1, group C, member 3) is a type II nuclear receptor that in humans is encoded by the PPARG gene. PPAR-gamma is one of a subfamily of closely related PPARs encoded by independent genes (Dreyer C et. al., 1992, Cell 68:879-887; Schmidt A et al., 1992, Mol. Endocrinol. 6:1634-1641; Zhu et al., 1993, J. Biol. Chem. 268:26817-26820; Kliewer S A et al., 1994, Proc. Nat. Acad. Sci. USA 91:7355-7359). Three mammalian PPARs have been isolated and termed PPAR-alpha, PPAR-gamma, and PPAR-delta (also known as NUC-1). These PPARs regulate expression of target genes by binding to DNA sequence elements, termed PPAR response elements (PPRE). To date, PPREs have been identified as the enhancers of a number of genes encoding proteins that regulate lipid metabolism, suggesting that PPARs play a role in the adipogenic signaling cascade and lipid homeostasis (Keller H et al., 1993, Trends Endocrin. Met. 4:291-296). PPARG has been associated with rescue of cognitive function in dementia and AD patients. Thus, activation of PPARG may confer a therapeutic benefit to patients an age-related cognitive disorder, dementia, or AD who are also being administered DHA. PPAR-gamma agonist as used herein is meant to include compounds or compositions which behave as agonists or partial agonists of the PPAR-gamma receptor. Suitable PPAR-gamma agonists for use with DHA treatment include, among others, prostaglandin J2, prostaglandin J2 analogues (e.g. 412-prostaglandin J2 and 15-deoxy-Δ12,14-prostaglandin J2), farglitazar, oxazolidinediones and thiazolidinediones. Exemplary thiazolidinediones include troglitazone, ciglitazone, pioglitazone, rosiglitazone, darglitazone and englitazone.

In some embodiments, the method described herein specifically excludes the administration, either adjunctively or not, of an NSAID, vitamin C, or Vitamin E, or combinations thereof.

In some embodiments, kits are provided for the methods described herein. In some embodiments, the kit comprises a molecular diagnostic test for the absence or presence of the ApoE4 allele, and a therapeutic amount of the DHA composition, such as in the dosage forms described herein. The kit may comprise single or multiple DHA composition dosage forms. The kit may further comprise instructions on various media, such as, among others, paper, audio or video tape, compact disc, memory cards, and digital video disc for carrying out the diagnostics test and for administration of the DHA. Where appropriate, the kit may further include dispensing devices for administration of the DHA, such as droppers, graduated syringes, and measuring cups.

### Example 1

### Study on Use of DHA for Treating Alzheimer's Disease

A clinical trial was carried out to determine whether chronic DHA supplementation slows the progression of cognitive and functional decline in human patients with mild to moderate Alzheimer's disease.

In the study, 402 individuals with mild to moderate Alzheimer's disease participated for 18 months at sites throughout the United States. Participants were randomized so that 60% of participants received approximately 2 grams of DHA (divided into 4 capsules—2 capsules taken twice a day), while 40% of the participants received an identical placebo of corn/soy oil. The DHA soft-gel capsules used in the study were provided by Martek Biosciences Corporation, and contained a microbial oil of 55% DHA (as a percentage of total fatty acids) in triglyceride form, in addition to tocopherol and orange flavoring. The capsules contained no detectable EPA. The placebo was a 50/50 mixture of corn/soy oil and also included mixed tocopherols, ascorbyl, palmitate, orange flavoring, and orange masking agent.

An initial screening was carried out to determine eligibility for the study. Inclusion criteria were as follows:

(1) male or female;

(2) 50 years of age or older; residing in the community at baseline (included assisted living facilities, but excluded long-term care nursing facilities);

(3) MMSE score at initial screening of from 14-26 (inclusive);

(4) no medical contraindications to study participation;

(5) fluent in English or Spanish;

(6) corrected vision and hearing sufficient for compliance with testing procedures

(7) supervision available for study medication;

(8) caregiver/study partner to accompany participant to all visits;

(9) study partner must have direct contact with the participant more than 2 days per week;

(10) able to ingest oral medication;

(11) daily DHA consumption less than or equal to 200 mg/day in prior two months estimated by an abbreviated DHA food frequency questionnaire;

(12) neuroimaging consistent with the diagnosis of Alzheimer's disease at some time after the onset of the memory decline;

(13) clinical laboratory values (no specific cutoffs or ranges were included in the protocol) were within normal limits or, if abnormal, were judged to be clinically insignificant by the investigator; and

(14) stable use of cholinesterase inhibitors and memantine is permitted if doses are stable for 4 months prior to enrollment.

Exclusion criteria were as follows:

(1) non-Alzheimer's disease dementia;

(2) residence in a long-term care facility at baseline;

(3) history of clinically significant stroke;

(4) modified Hachinski Ischemia score ≧4;

(5) current evidence or history in past two years of epilepsy, seizure, focal brain lesion, head injury with loss of consciousness or DSM IV criteria for any major psychiatric disorder including psychosis, major depression, bipolar disorder, alcohol or substance abuse;

(5) sensory impairment which would prevent subject from participating in or cooperating with the protocol;

(6) use of another investigational agent within two months;

(7) evidence of any significant clinical disorder or laboratory finding that renders the participant unsuitable for receiving an investigational new drug including clinically significant or unstable hematologic, hepatic, cardiovascular (including history of ventricular fibrillation or ventricular tachycardia), pulmonary, gastrointestinal, endocrine, metabolic, renal, or other systemic disease or laboratory abnormality; and

(8) active neoplastic disease (skin tumors other than melanoma may be included; participants with stable prostate cancer could have been included at the discretion of the Project Director).

Following the eligibility assessment, 238 of the 402 patients were randomly assigned to the DHA treatment group and 164 patients to the placebo group. Of the 238 patient DHA treatment group, 171 patients completed the full 18 month course of treatment. Of the 164 placebo group, 124 patients completed the full 18 month course of treatment.

Baseline statistics for the total patient population, the placebo group, and the DHA group, are shown in Table 10 below.

TABLE 10

| | TOTAL | | | |
|---|---|---|---|---|
| | STUDY | PLACEBO | | |
| | POPULATION | GROUP | DHA GROUP | |
| | (N = 402) | (N = 164) | (N = 238) | P |
| AGE | 76 ± 8.7 | 76 ± 7.8 | 76 ± 9.3 | NS |
| % FEMALE | 52% | 60% | 47% | 0.015 |
| EDUCATION | 14 ± 2.8 | 14 ± 2.7 | 14 ± 2.9 | NS |
| APOE4 | 57.7% | 57.9% | 57.6% | NS |
| MMSE | 20.67 ± 3.6 | 20.3 ± 3.7 | 20.9 ± 3.6 | 0.095 |
| ADAS COG | 23.85 ± 9 | 23.96 ± 9.2 | 23.77 ± 8.9 | NS |
| CDR-SOB | 5.68 ± 2.61 | 5.77 ± 2.61 | 5.61 ± 2.62 | NS |
| PLASMA | 3.16 ± 1.12 | 3.13 ± 0.96 | 3.18 ± 1.21 | NS |
| DHA | | | | |
| CEI | 85.8% | 83.5% | 87.4% | NS |
| MEMANTINE | 60.45% | 63.4% | 58.4% | NS |

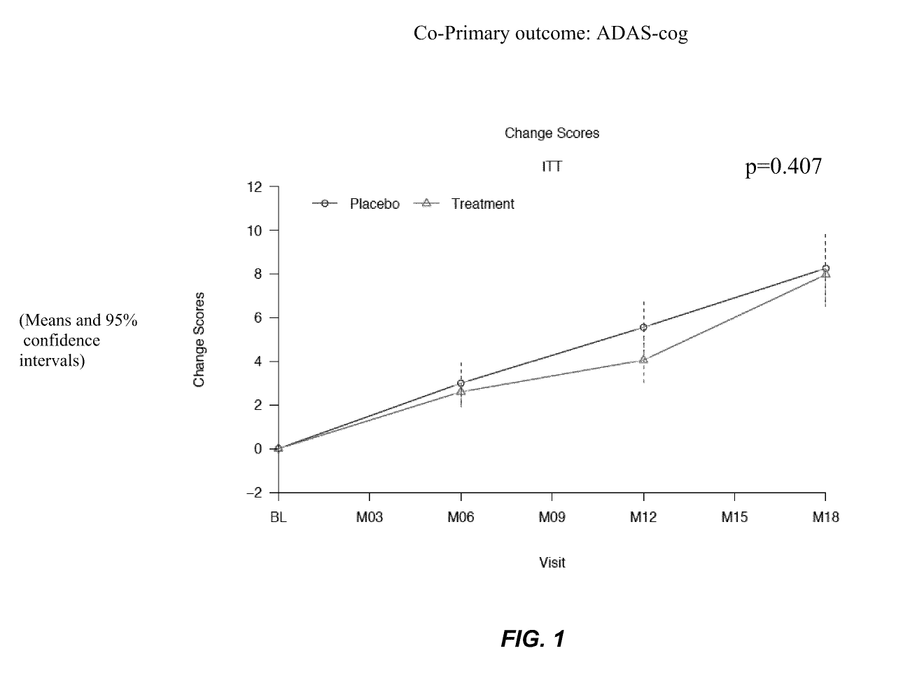

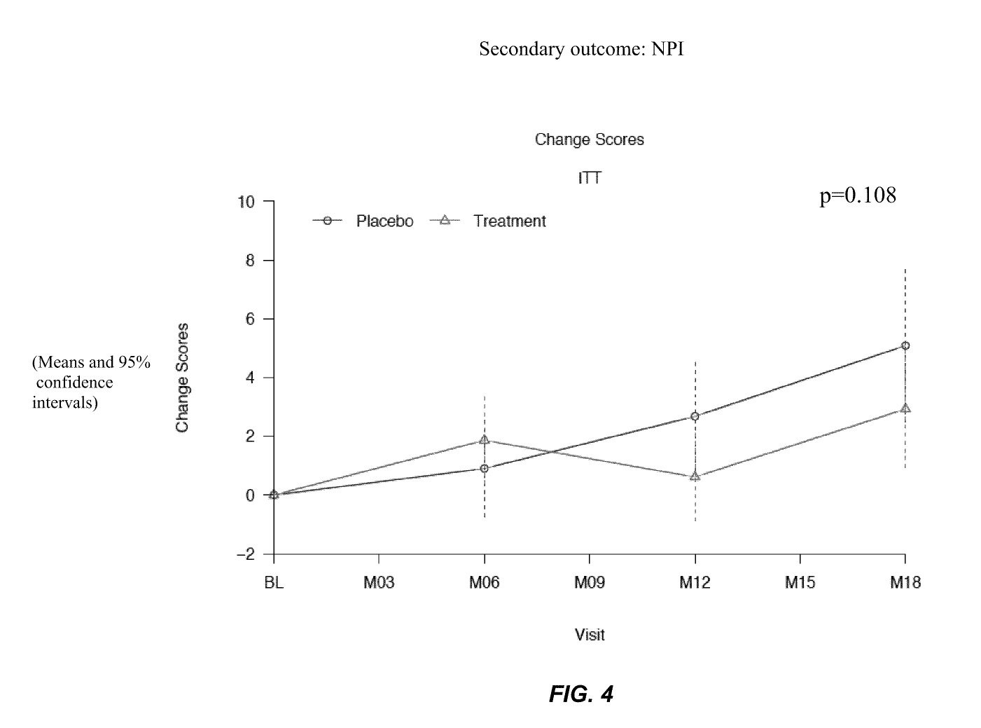

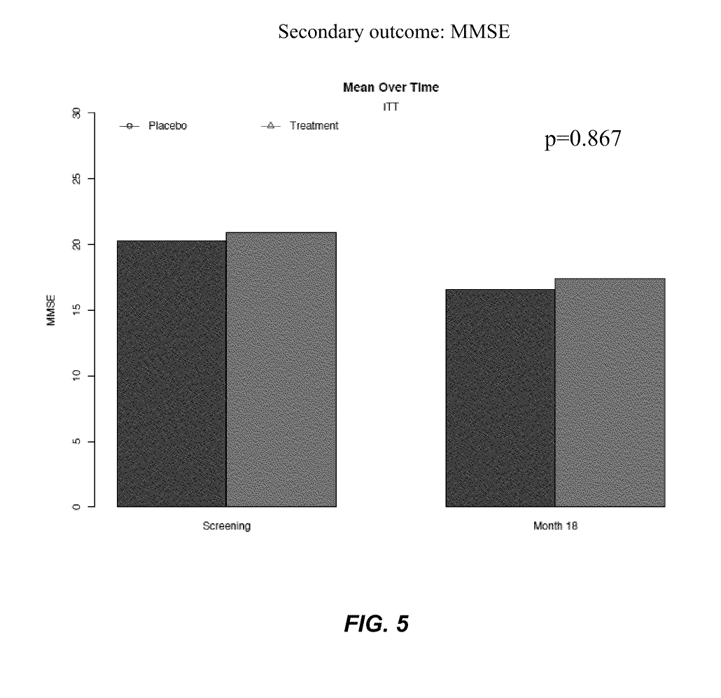

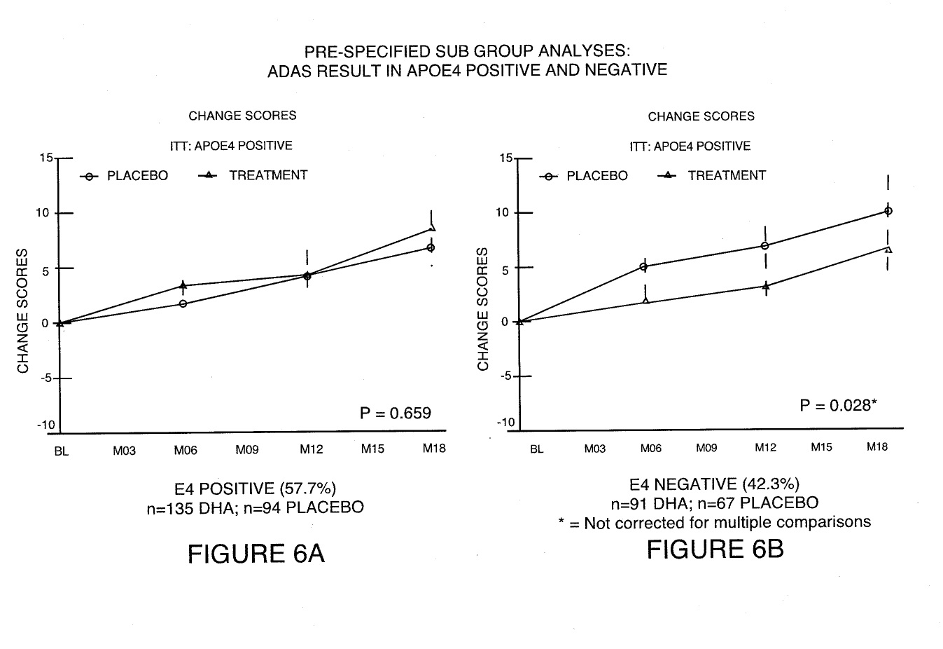

Various parameters were measured at baseline and at every 6 months through the conclusion of the trial at 18 months. Results from those measurements are set forth in FIGS. 1 through 6.

Vital Signs and Lab Results: There was a modest decline in diastolic blood pressure, heart rate, and triglycerides. There was a modest increase in cholesterol and LDL levels. There was no change in weight, systolic blood pressure, or HDL levels.

All subjects without contraindication to cerebrospinal fluid (CSF) exam (e.g., anticoagulation) were invited to participate in the CSF study. In these subjects, lumbar puncture was performed in the morning after an overnight fast.

Plasma phospholipid fatty acid levels were determined using established methods, with modifications for cerebrospinal fluid analysis (Arterburn et al., 2007, Lipids 42(11):1011-1024; Arterburn et al., 2008, J Am Diet Assoc. 108(7):1204-1209). The fatty acid profiles were expressed as a percent of the total μg of fatty acid (weight percent).

Plasma phospholipid DHA increased in the DHA treatment group from 3.18 wt % at baseline to 9.1 wt % at 6 months, 10.23 wt % at 12 months, and 9.77 wt % at 18 months (p<0.001) with no significant change in plasma phospholipid DHA in the placebo group (3.13 at baseline, 3.12 at 18 months). In a sub-group of 44 subjects volunteering for CSF collection at baseline and 18 months (n=29 DHA, n=15 placebo), a significant increase in CSF DHA was observed in the DHA supplemented group (2.53 wt % at baseline, 3.45 wt % at 18 months (p<0.001) but not in the placebo group (2.50 wt % at baseline, 2.17 wt % at 18 months).

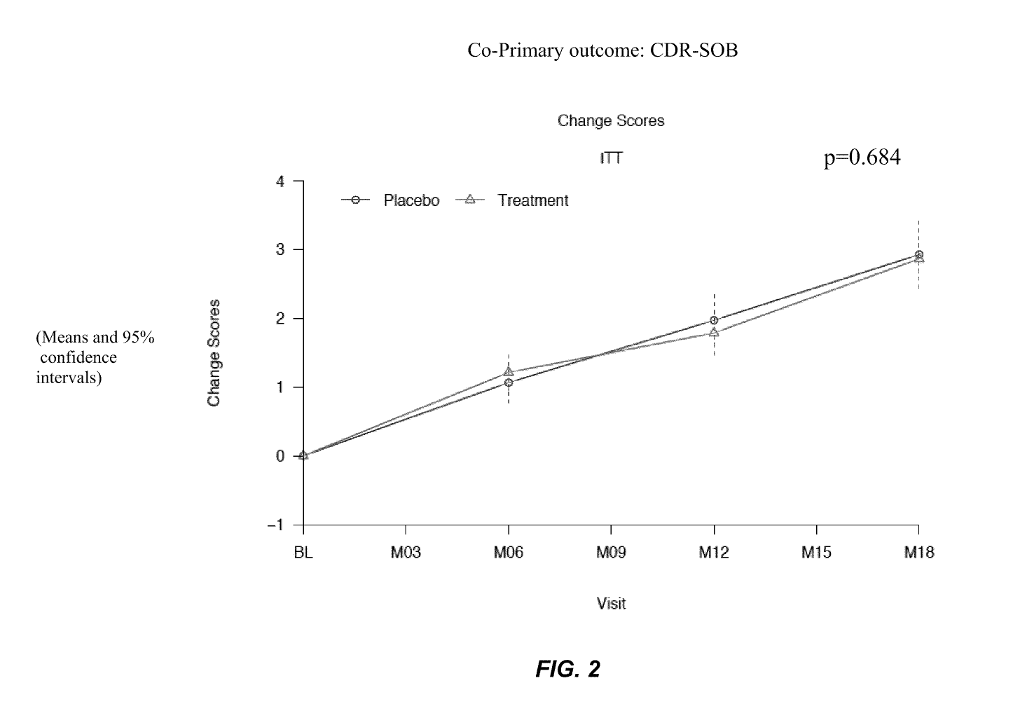

Co-primary outcome measures: The rate of change on ADAS-cog did not differ between treatment groups (8.27±8.9 points change, unadjusted, over 18 months for DHA compared to 7.98±9.84 points for placebo; p=0.41). The rate of change on CDR-SOB also did not differ between treatment groups (2.93±2.83 points change over 18 months for DHA compared to 2.87±2.93 points for placebo; p=0.68) (FIG. 2b). Confirmatory GEE and ANCOVA analyses and an ad hoc LME analysis including both gender and baseline MMSE as covariates also failed to show evidence of a benefit of DHA treatment.

Secondary outcome measures: The LME analysis revealed no difference between DHA and placebo in rate of decline on ADCS-ADL (11.51±13.23 points change over 18 months for DHA compared to 10.43±11.74 points for placebo; p=0.38) (FIG. 2c) or NPI (2.93±13.62 points change over 18 months for DHA compared to 5.09±15.08 points for placebo; p=0.11) (FIG. 2d). An ANCOVA analysis showed no difference between treatment groups in change of MMSE from baseline to 18 months (−3.70±4.95 points change over 18 months for DHA compared to −4.0±4.7 points for placebo; p=0.88).

Among the subjects participating in the MRI sub-study (n=53 DHA, n=49 placebo), an ANCOVA analysis showed no evidence of an effect of DHA treatment upon the absolute amount of volume change over 18 months for total brain (24.7±12.3 cm³ in DHA, 24.0±14.6 cm³ in placebo, p=0.79), hippocampus (left hippocampus: 141±104 mm³ in DHA, 175±135 mm³ in placebo; p=0.17); right hippocampus 176±128 mm³ in DHA, 148±109 mm³ in placebo; p=0.29)), or total ventricular volume (9.1±5.0 cm³ in DHA, 8.1±5.9 cm³ in placebo, p=0.55).

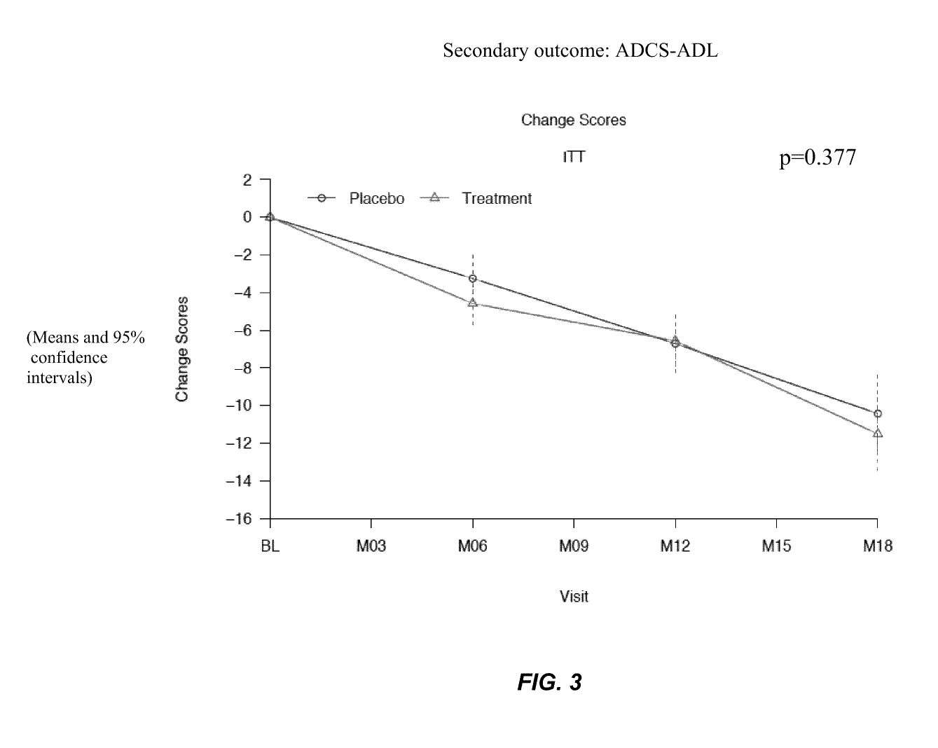

A pre-planned secondary analysis was also carried out examining the effect of DHA treatment and ApoE4 (E4) allelic status. While there was no DHA treatment effect on any outcome measure in the E4-positive group, there was an effect on the ADAS-cog favoring DHA treatment in the E4-negative group (6.23±8.58 points change over 18 months for 61 DHA subjects compared to 10.11±10.58 points for 48 placebo subjects; p=0.028) (FIG. 3a, 3b). This effect was also evident on the MMSE (−3.36±4.78 in DHA compared to 5.12±5.08 in placebo; p=0.034), but was not present on the CDR-SOB, the ADCS-ADL, or the NPI. Neither was an effect of DHA seen upon rates of brain atrophy among ApoE4 negative subjects participating in the MRI sub-study (n=21 DHA, n=17 placebo).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of treating an age-related cognitive disorder, comprising administering to a human subject in need thereof who is identified as being negative for the ApoE4 allele an effective amount of a composition comprising docosahexaenoic acid (DHA) to treat the age-related cognitive disorder, wherein the composition has a DHA to eicosapentaenoic acid (EPA) ratio higher than 4:1 wt/wt or has no EPA.

2. A method of treating an age-related cognitive disorder, comprising:
(a) identifying a human subject negative for the ApoE4 allele; and
(b) administering to the human subject in need thereof an effective amount of a composition comprising docosahexaenoic acid (DHA) to treat the age-related cognitive disorder, wherein the composition has a DHA to eicosapentaenoic acid (EPA) ratio higher than 4:1 wt/wt or has no EPA

3. The method of claim 1 in which the age-related cognitive disorder is mild cognitive impairment (MCI), age-related cognitive decline (ARCD), age-associated memory impairment (AAMI), or age-associated cognitive impairment (AACI).

4. A method of treating dementia, comprising administering to a human subject in need thereof who is identified as being negative for the ApoE4 allele an effective amount of a composition comprising docosahexaenoic acid (DHA) to treat the dementia, wherein the composition has a DHA to eicosapentaenoic acid (EPA) ratio higher than 4:1 wt/wt or has no EPA.

5. A method of treating dementia, comprising:
(a) identifying a human subject negative for the ApoE4 allele; and
(b) administering to the human subject in need thereof an effective amount of a composition comprising docosahexaenoic acid (DHA) to treat the dementia, wherein the composition has a DHA to eicosapentaenoic acid (EPA) ratio higher than 4:1 wt/wt or has no EPA.

6. A method of treating Alzheimer's disease, comprising administering to a human subject in need thereof who is identified as being negative for the ApoE4 allele an effective amount of a composition comprising docosahexaenoic acid (DHA) to treat the Alzheimer's disease, wherein the composition has a DHA to eicosapentaenoic acid (EPA) ratio higher than 4:1 wt/wt or has no EPA.

7. A method of treating Alzheimer's disease, comprising:
(a) identifying a human subject negative for the ApoE4 allele; and
(b) administering to the human subject in need thereof an effective amount of a composition comprising docosahexaenoic acid (DHA) to treat the Alzheimer's disease, wherein the composition has a DHA to eicosapentaenoic acid (EPA) ratio higher than 4:1 wt/wt or has no EPA.

8. The method of claim 1 in which the DHA to EPA ratio is at least 5:1 wt/wt.

9. The method of claim 1 in which the DHA to EPA ratio is at least 10:1 wt/wt.

10. The method of claim 1 in which the DHA to EPA ratio is at least 20:1 wt/wt.

11. The method of claim 1 in which the DHA to EPA ratio is about 16:1 wt/wt.

12. The method of claim 1 in which the composition of DHA is substantially free of EPA.

13. The method of claim 1 in which the composition of DHA has no EPA.

14. The method of claim 1 in which the DHA is at least 40 wt % of total wt of fatty acid content.

15. The method of claim 1 in which the DHA is at least 50 wt % of total wt of fatty acid content.

16. The method of claim 1 in which the DHA is at least 90 wt % of total wt of fatty acid content.

17. The method of claim 1 in which the DHA is at least 99 wt % of total wt of fatty acid content.

18. The method of claim 1 in which the composition of DHA is a microbial oil or is derived from microbial oil.

19. The method of claim 18 in which the microbial oil is from Crypthecodinium, Schizochytrium, or Thraustochytrium.

20. The method of claim 1 in which the DHA is in the form of a phospholipid.

21. The method of claim 1 in which the DHA is in the form of a triglyceride.

22. The method of claim 1 in which the DHA is in the form of a free fatty acid.

23. The method of claim 1 in which the DHA is in the form of an alkyl ester.

24. The method of claim 23 in which the DHA alkyl ester is DHA methyl ester, ethyl ester, or propyl ester.

25. The method of claim 1 in which the DHA is administered adjunctively with an anti-Alzheimer's drug.

26. The method of claim 25 in which the anti-Alzheimer's drug is administered sequentially.

27. The method of claim 25 in which the anti-Alzheimer's drug is administered simultaneously.

28. The method of claim 25 in which the anti-Alzheimer's drug is an acetylcholinesterase inhibitor.

29. The method of claim 28 in which the acetylcholinesterase inhibitor is selected from tacrine, donepezil, rivastigmine, and galantamine.

30. The method of claim 25 in which the anti-Alzheimer's drug is an NMDA receptor antagonist.

31. The method of claim 30 in which the NMDA receptor antagonist is memantine.

32. The method of claim 25 in which the anti-Alzheimer's drug is a vaccine.

33. The method of claim 32 in which the vaccine is a β-amyloid vaccine.

34. The method of claim 25 in which the anti-Alzheimer's drug is an antibody against β-amyloid protein.

35. The method of claim 34 in which the antibody comprises a monoclonal antibody against β-amyloid protein.

36. The method of claim 35 in which the monoclonal antibody is a humanized monoclonal antibody.

37. The method of claim 25 in which the anti-Alzheimer's drug is a β or γ-secretase inhibitor.

38. The method of claim 1 in which the composition of DHA is administered adjunctively with an anti-inflammatory agent.

39. The method of claim 38 in which the anti-inflammatory agent is selected from a nonsteroidal anti-inflammatory drug (NSAID) or a steroidal anti-inflammatory drug.

40. The method of claim any of claim 1 in which the composition of DHA is administered with a cholesterol lowering agent.

41. The method of claim 40 in which the cholesterol lowering agent is selected from, bile acid binding resins; fibric acid derivatives; and statin compounds.

42. The method of claim 1 in which the subject carries the ApoE2 or ApoE3 allele.

43. The method of claim 42 in which the subject is homozygous for the ApoE2 or ApoE3 allele.

44. The method of claim 1 in which the composition further comprises an additional unsaturated lipid.

45. The method of claim 44 in which the unsaturated lipid is a polyunsaturated lipid.

46. The method of claim 45 in which the polyunsaturated lipid is an omega-3 or omega-6 fatty acid.

47. The method of claim 46 in which the omega-6 fatty acid is docosapentaenoic acid (DPA).

48. The method of claim 1 in which the composition further comprises vitamin E.

49. The method of claim 48 in which the vitamin E is a tocopherol.

50. The method of claim 49 in which the tocopherol is selected α, β, γ and δ tocopherol, or combinations thereof.

51. The method of claim 1 in which the DHA is administered in an amount of from about 1.5 mg per kg body weight per day to about 125 mg per kg body weight per day.

52. The method of claim 1 in which the DHA is administered in an amount of from about 150 mg to about 10 g per day.

53. The method of claim 1 in which the DHA is administered in an amount of from about 0.5 g per day to about 5 g per day.

54. The method of claim 1 in which the DHA is administered in an amount of from about 1 g per day to about 5 g per day.

55. The method of claim 1 in which the DHA is administered in an amount of about 1 g per day.

56. The method of claim 51 in which the DHA is administered at least once per day.

57. The method of claim 51 in which the DHA is administered at least twice per day.

58. The method of claim 51 in which the DHA is administered at least two times weekly.

59. The method of claim 1 in which the DHA is administered for at least 6 months.

60. The method of claim 1 in which the DHA is administered for at least 1 yr.

61. The method of claim 1 in which the DHA is administered for at least 1.5 yrs.

62. The method of claim 1 in which the DHA is administered for at least 2 yrs.

63. The method of claim 1 in which the DHA is administered for at least 5 yrs.

64. The method of claim 1 in which the composition is administered in the form of a capsule, gel, tablet, or emulsion.

65. The method of claim 1 in which the DHA composition is administered in the form a gelatin capsule selected from the group consisting of: porcine gelatin capsules, bovine gelatin capsules, vegetarian gelatin capsules, and alginate gelatin capsules.

66. The method of claim 1 in which the DHA is administered orally.

67. The method of claim 1 in which the subject is at an increased risk for developing Alzheimer's disease.

68. The method of claim 1 which includes the step of testing a human subject for presence or absence of the ApoE4 allele.

69. A method of treating a human subject suffering from mild to moderate Alzheimer's disease, comprising:
(a) identifying a human subject negative for the ApoE4 allele; and
(b) administering to the human subject in need thereof an oral dosage formulation comprising fatty acids wherein the formulation comprises at least about 40% DHA, by weight of the total fatty acid content of the formulation, wherein the amount of DHA administered to the subject in need thereof is from 860 mg up to about 6 g of DHA, wherein the formulation is provided in the substantial absence of EPA.

70. The method as recited in claim 69 wherein the formulation has no detectable EPA.

71. The method as recited in claim 69 wherein the DHA comprises at least 50 wt % of total wt of fatty acid content.

72. The method as recited in claim 70 wherein the DHA comprises at least 50 wt % of total wt of fatty acid content.

73. The method as recited in claim 71 wherein the DHA comprises at least 90 wt % of total wt of fatty acid content.

74. The method as recited in claim 72 wherein the DHA comprises at least 90 wt % of total wt of fatty acid content.

75. The method of claim 69 wherein the DHA is in the form of a triglyceride.

76. The method of claim 69 wherein the DHA is in the form of a free fatty acid.

77. The method of claim 69 wherein DHA is in the form of an alkyl ester.

78. The method as recited in claim 77 wherein the DHA alkyl ester is DHA methyl ester, ethyl ester, or propyl ester.

79. The method of claim 69, wherein the human subject suffers from mild Alzheimer's disease.

80. A method of treating a human subject suffering from mild to moderate Alzheimer's disease, comprising:
(a) identifying a human subject negative for the ApoE4 allele; and
(b) administering to the human subject in need thereof an oral dosage formulation comprising DHA in an amount sufficient to raise the plasma phospholipid DHA levels at about at least 3 fold in six months wherein the formulation is provided in the substantial absence of EPA.

81. A method of treating a human subject suffering from mild to moderate Alzheimer's disease, comprising:
(a) identifying a human subject negative for the ApoE4 allele; and
(b) administering to the human subject in need thereof an oral dosage formulation comprising DHA in an amount sufficient to raise the cerebrospinal fluid DHA levels by at least 30% wherein the formulation is provided in the substantial absence of EPA.
